(19) United States
(12) Patent Application Publication
(10) Pub. No.: US 2019/0388515 A1
(43) Pub. Date: Dec. 26, 2019

(54) INJECTABLE SOLUTION AT PH 7 COMPRISING AT LEAST ONE BASAL INSULIN THE PI OF WHICH IS FROM 5.8 TO 8.5 AND A CO-POLYAMINO ACID BEARING CARBOXYLATE CHARGES AND HYDROPHOBIC RADICALS

(71) Applicant: ADOCIA, Lyon (FR)

(72) Inventors: Alexandre GEISSLER, Lyon (FR); You-Ping CHAN, Ternay (FR); Romain NOEL, Villeurbanne (FR); Richard CHARVET, Rillieux La Pape (FR); Nicolas LAURENT, Miribel (FR)

(73) Assignee: ADOCIA, Lyon (FR)

(21) Appl. No.: 16/563,027

(22) Filed: Sep. 6, 2019

Related U.S. Application Data
(63) Continuation-in-part of application No. 16/213,809, filed on Dec. 7, 2018.
Application No. 16/213,809 is a continuation-in-part of application No. 16/212,960, filed on Dec. 7, 2018.
(60) Provisional application No. 62/606,138, filed on Dec. 7, 2017.

(30) Foreign Application Priority Data
Dec. 7, 2017 (FR) 17/61807
Jun. 29, 2018 (FR) 18/55934
Jun. 29, 2018 (FR) 18/56067

(51) Int. Cl. A61K 38/28; A61K 9/08; A61K 9/00; A61K 47/42

(52) U.S. Cl. CPC A61K 38/28; A61K 47/42; A61K 9/0019; A61K 9/08

(57) ABSTRACT

Described are physically stable compositions in the form of an injectable aqueous solution, the pH of which is from 6.0 to 8.0, comprising at least a basal insulin of which the isoelectric point (pI) is from 5.8 to 8.5, and a co-polyamino acid bearing carboxylate charges and at least one hydrophobic radical.

This application is a continuation-in-part application of U.S. application Ser. No. 16/212,960 filed Dec. 7, 2018, and U.S. application Ser. No. 16/213,809 filed Dec. 7, 2018, the entire disclosure of each of which is incorporated herein by reference in its entirety.

The invention relates to the therapies by injection of insulin(s) for treating diabetes.

The invention relates to physically stable compositions in the form of an injectable aqueous solution, the pH of which is from 6.0 to 8.0, comprising at least one basal insulin of which the isoelectric point (pI) is from 5,8 to 8.5 and a co-polyamino acid bearing carboxylate charges and hydrophobic radicals.

Insulin therapy, or the therapy for diabetes by injection of insulin, has undergone remarkable progress in recent years, in particular thanks to the development of novel insulins which offer a better correction of the glycemia of patients in comparison to human insulin, and which make it possible to better simulate the physiological activity of the pancreas.

When type II diabetes is diagnosed in a patient, a gradual treatment is implemented. First, the patient takes oral antidiabetic drugs (OAD) such as metformin. When the OAD alone are no longer sufficient for regulating the glucose level in the blood, a change in treatment must be made, and, depending on the specificities of the patients, different combinations of treatments can be implemented. For example, the patient can have a treatment based on a basal insulin of the insulin glargine or insulin detemir type in addition to the OAD, and then, depending on the development of the pathology, a treatment based on basal insulin and on prandial insulin.

Moreover, today, in order to ensure the transition of the treatments with OAD to a basal insulin/prandial insulin treatment when the former treatments are no longer able to control the blood glucose level, the injection of analogs of GLP-1 RA is recommended.

The GLP-1 RA, standing for glucagon-like peptide-1 receptor agonists, are insulinotropic peptides or incretins and belong to the family of the gastrointestinal hormones (or gut hormones) which stimulate the secretion of insulin when the glycemia is too high, for example, after a meal.

The gastrointestinal hormones (gut hormones) are also referred to satiety hormones. They comprise, in particular, GLP-1 RA (glucagon-like peptide-1 receptor agonist) and GIP (glucose-dependent insulinotropic peptide), oxyntomodulin (a derivative of proglucagon), peptide YY, amylin, cholecystokinin, pancreatic polypeptide (PP), ghrelin and enterostatin which have peptide or protein structures. They also stimulate the secretion of insulin, in response to glucose and fatty acids, and, as such, they are potential candidates for the treatment of diabetes.

Among said gastrointestinal hormones, GLP-1 RA are those that, to date, have provided the best results in the development of drugs. They have made it possible for patients suffering from type II diabetes to lose weight while at the same time having a better control of their glycemia.

Analogs or derivatives of GLP-1 RA have also been developed, in particular in order to improve their stability.

Moreover, a diabetic patient, to cover his/her daily insulin needs, currently has available, in a simplified manner, two types of insulins having complementary actions: the prandial insulins (or so-called rapid-acting insulins) and the basal insulins (or so-called short-acting insulins).

The prandial insulins allow a rapid management (metabolization and/or storage) of the glucose ingested in meals and snacks. The patient must self-administer an injection of a prandial insulin before each ingestion of food, namely approximately 2 to 3 injections a day. The prandial insulins used the most are: human recombinant insulin, Novolog® (insulin aspart from NOVO NORDISK), Humalog® (insulin lispro from ELI LILLY) and Apidra® (insulin glulisine from SANOFI).

The basal insulins ensure the maintenance of the glycemic homeostasis of the patient outside periods of food intake. They act essentially by blocking the endogenous glucose production (hepatic glucose). The daily dose of basal insulin generally corresponds to 40-50% of the total daily insulin needs. Depending on the basal insulin used, this dose is administered in 1 or 2 injections distributed regularly over the course of the day. The most used basal insulins are Levemir® (insulin detemir from NOVO NORDISK) and Lantus® (insulin glargine from SANOFI).

For the sake of completeness, it should be noted that NPH (insulin NPH standing for neutral protamine Hagedorn insulin; Humuline NPH®, Insulatard®) is the oldest basal insulin. This formulation is the result of a precipitation of human insulin (anionic at neutral pH) by a cationic protein, the protamine. The microcrystals thus formed are dispersed in an aqueous suspension and dissolve slowly after subcutaneous injection. This slow dissolution ensures a prolonged release of the insulin. However, this release does not ensure a constant insulin concentration over time. The release profile is bell-shaped and lasts only from 12 to 16 hours. Therefore, said insulin is injected twice daily. This basal insulin NPH here is much less effective than the modern basal insulins. Levemir® and Lantus®. NPH is an intermediate-acting basal insulin.

The principle of NPH has evolved with the appearance of the rapid insulin analogs giving rise to so-called “premix” products that offer rapid action and intermediate action simultaneously. NovoLog Mix® (NOVO NORDISK) and Humalog Mix® (ELI LILLY) are formulations comprising a rapid insulin analog, Novolog® and Humalog®, complexed partially by the protamine. These formulations thus contain microcrystals of insulin analog, the action of which is referred to as intermediate, and, a portion of insulin that has remained soluble, the action of which is rapid. These formulations indeed offer the advantage of a rapid insulin, but they also have the defect of NPH, that is to say a duration of action limited to from 12 to 16 hours and a “bell” shaped insulin release profile. However, these products make it possible for the patient to self-administer a single injection of an intermediate-acting basal insulin together with a rapid-acting prandial insulin. However, many patients want to reduce their number of injections.

The currently marketed basal insulins can be classified based on the technical solution that makes it possible to obtain the prolonged action, and, to date, two approaches are used.

The first approach, that of insulin detemir, is the in vivo binding to albumin. This is an analog, soluble at pH 7, which comprises a side chain of fatty acid (tetradecanoyl) bound at position B29 which, in vivo, allows this insulin to associate with albumin. Its prolonged action is primarily due to this affinity for albumin after subcutaneous injection.

However, its pharmacokinetic profile cannot cover a day, and as a result it is usually used in two injections daily.

Another insulin which is soluble at pH 7 is insulin degludec marketed under the name of Tresiba®d. It also comprises a side chain of fatty acid bound to the insulin (hexadecandioyl-γ-L-Glu).

The second approach, that of insulin glargine, is the precipitation at physiological pH. Insulin glargine is an analog of human insulin obtained by elongation of the C-terminal part of the B chain of human insulin by two arginine residues and by substitution of the asparagine residue A21 with a glycine residue (U.S. Pat. No. 5,656,722). The addition of two arginine residues was intended to adjust the pI (isoelectric point) of insulin glargine at physiological PH, and, in this manner, to make this analog of human insulin insoluble in a physiological medium.

In addition, the substitution of A21 was intended to make the insulin glargine stable at acidic pH and to be able to formulate it in the form of a solution which is an injectable solution at acidic pH. During the subcutaneous injection, the passage of insulin glargine from an acidic pH (pH 4-4.5) to a physiological pH (neutral pH) causes its precipitation under the skin. The slow redissolution of the insulin glargine microparticles ensures a slow and prolonged action.

The hypoglycemic effect of insulin glargine is nearly constant for a duration of 24 hours, which alloy's most of the patients to only inject themselves once a day.

Insulin glargine is considered today to be the most used basal insulin.

However, the necessarily acidic pH of the formulations of basal insulins, the isoelectric point of which is from 5.8 to 8.5, of the insulin glargine type can be a real disadvantage, since this acidic pH of the formulation of insulin glargine sometimes causes pain to the patients during the injection and especially it stops any formulation with other proteins, particularly with the prandial insulins, since the latter are not stable at acidic pH. The impossibility of formulating a prandial insulin at acidic pH is due to the fact that, under these conditions, a prandial insulin undergoes a side reaction of deamidation in position A21, which does not allow it to meet the stability requirements applicable to injectable drugs.

To date, in the applications WO 2013/021143 A1, WO 2013/104861 A1, WO 2014/124994 A1 and WO 2014/124993 A1, it was demonstrated that it was possible to solubilize these basal insulins of the insulin glargine type, the isoelectric point of which is from 5.8 to 8.5, at neutral pH, while maintaining a difference in solubility between the in-vitro medium (the container) and the in-vivo medium (under the skin) regardless of the pH.

In particular, the application WO 2013/104861 A1 describes compositions in the form of an injectable aqueous solution, the pH of which is from 6.0 to 8.0, comprising at least (a) a basal insulin, the isoelectric point pI of which is from 5.8 to 8.5, and (b) a co-polyamino acid bearing carboxylate charges and substituted with hydrophobic radicals.

These compositions of the prior art have the main disadvantage that they are not sufficiently stable to meet the specifications applicable to the pharmaceutical formulations.

In the examples of the experimental part of the present patent application, it is demonstrated that the compositions described, in particular, in WO 2013/104861 A1 present an unsatisfactory stability over time.

Therefore, there is a need to find a solution which makes it possible to dissolve a basal insulin of which the isoelectric point (pI) is from 5.8 to 8.5, while preserving the basal profile thereof after injection, but which also makes it possible to meet the standard physical stability conditions for the pharmaceutical products based on insulin.

Surprisingly, the applicant has found that the co-polyamino acids bearing carboxylate charges and hydrophobic radicals according to the invention make it possible to obtain compositions in the form of solutions which not only meet the requirements described in WO 2013/104861 A1, but which, in addition, are capable of conferring an improved physical stability to said compositions without the need to increase the quantity of excipients used.

These performances never reached a priori are, in addition, maintained when the basal insulin of which the isoelectric point is from 5.8 to 8.5, is combined in the composition with a prandial insulin and/or a gastrointestinal hormone.

Thus, surprisingly, the affinity of the co-polyamino acids according to the invention for insulin glargine has been increased, in that it makes it possible to obtain a solubilization and a stabilization of the solutions of insulin glargine at an [Hy]/[basal insulin] ratio lower that of the prior art; in addition, these results are obtained without alterating, and even with an improvement, of the propensity of insulin glargine to precipitate, as demonstrated in the experimental part.

This improvement of the affinity moreover makes it possible to limit the level of exposure to said excipients in the context of chronic treatments.

The co-polyamino acids bearing carboxylate charges and hydrophobic radicals Hy according to the invention present an excellent resistance to hydrolysis. This can be verified, in particular, under accelerated conditions, for example, by hydrolysis tests at basic pH (pH 12).

In addition, forced oxidation tests, for example, of the Fenton oxidation type, show that the co-polyamino acids bearing carboxylate charges and hydrophobic radicals Hy exhibit a good resistance to oxidation.

The invention thus relates to physically stable compositions in the form of an injectable aqueous solution, the pH of which is from 6.0 to 8.0, comprising at least:
a) one basal insulin, the isoelectric point (pI) of which is from 5.8 to 8.5, and
b) a co-polyamino acid bearing carboxylate charges and at least one hydrophobic radical according to formula I.

In an embodiment, the invention relates to a composition in the form of an injectable aqueous solution, the pH of which is from 6.0 to 8.0, comprising at least:
a) one basal insulin, the isoelectric point pI of which is from 5.8 to 8.5;
b) a co-polyamino acid bearing carboxylate charges and hydrophobic radicals Hy, said co-polyamino acid consisting of glutamic or aspartic units, and said hydrophobic radicals Hy having the following formula I:

(GpR)ᵣ(GpA)ₐ (GpC)ₚ Formula I

in which
GpR is a radical according to formula II or II′:

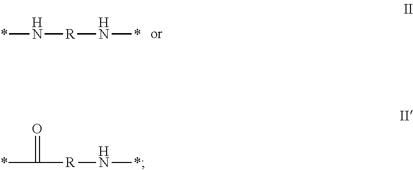

GpA is a radical according to formula III or III′:

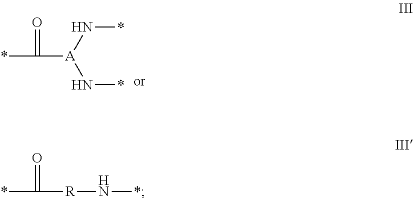

GpC is a radical according to formula IV:

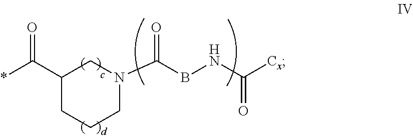

the * indicates the sites of attachment of the different groups bound by amide functions;
a is an integer to 0 or 1;
b is an integer to 0 or 1;
p is an integer to 1 or 2, and
   if p is equal to 1, then a is equal to 0 or 1 and GpA is a radical according to formula III′, and
   if p is equal to 2, then a is equal to 1 and GpA is a radical according to formula III;
c is an integer equal to 0 or 1, and, if c is equal to 0, then d is equal to 1 or 2;
d is an integer equal to 0, to 1 or 2;
r is an integer equal to 0 or 1, and
   if r is equal to 0, then the hydrophobic radical according to formula I is bound to the polyamino acid via a covalent bond between a carbonyl of the hydrophobic radical and a nitrogen atom in N-terminal position of the co-polyamino acid, thus forming an amide function resulting from the reaction of an amine function in N-terminal position of the precursor of the co-polyamino acid and an acid function borne by the precursor of the hydrophobic radical, and
   if r is equal to 1, then the hydrophobic radical according to formula I is bound to the co-polyamino acid:
      via a covalent bond between a nitrogen atom of the hydrophobic radical and a carbonyl of the co-polyamino acid, thus forming an amide function resulting from the reaction of an amine function of the precursor of the hydrophobic radical and an acid function borne by the precursor of the co-polyamino acid, or
      via a covalent bond between a carbonyl of the hydrophobic radical and a nitrogen atom in N-terminal position of the co-polyamino acid, thus forming an amide function resulting from the reaction of an acid function of the precursor of the hydrophobic radical and an amine function in N-terminal position borne by the precursor of the co-polyamino acid;
R is a radical chosen from the group consisting of:
   a linear or branched divalent alkyl radical comprising, if GpR is a radical according to formula II, from 2 to 12 carbon atoms, or, if GpR is a radical according to formula II′, from 1 to 11 carbon atoms;
   a linear or branched divalent alkyl radical comprising, if GpR, is a radical according to formula II, from 2 to 11 carbon atoms, or, if GpR is a radical according to formula II′, from 1 to 11 carbon atoms, said alkyl radical bearing one or more —CONH₂ functions, and
   an unsubstituted ether or polyether radical comprising from 4 to 14 carbon atoms and from 1 to 5 oxygen atoms;
A is a linear or branched alkyl radical comprising from 1 to 8 carbon atoms, such as 1 to 6 carbon atoms;
B is a linear or branched alkyl radical, optionally comprising an aromatic ring, comprising from 1 to 9 carbon atoms;
Cx is a linear or branched monovalent alkyl radical, in which x indicates the number of carbon atoms, and:
   if p is equal to 1, x is from 9 to 25 (9≤x≤25), such as from 11 to 25 (11≤x≤25);
   if p is equal to 2, x is from 9 to 15 (9≤x≤15),
the ratio i between the number of hydrophobic radicals and the number of glutamic or aspartic units being between 0<i≤0.5;
when several hydrophobic radicals are borne by a co-polyamino acid, then they are identical or different,
the degree of polymerization DP in glutamic or aspartic units is from 5 to 250;
the free acid functions being in the form of a salt of an alkaline cation chosen from the group consisting of Na⁺ and K⁺.

The pH of the compositions according to the invention is from 6.0 to 8.0, preferably from 6.6 to 7.8, or more preferably from 6.8 to 7.6.

In embodiments, Hy comprises less than 30 carbon atoms, such as from 15 to 30 carbon atoms.

Said co-polyamino acid bearing carboxylate charges and hydrophobic radicals Hy is soluble in an aqueous solution at a pH from 6.0 to 8.0, at a temperature of 25° C., and at a concentration of less than 60 mg/mL.

“Physically stable composition” is understood to mean compositions that satisfy the criteria of the visual inspection described in the European, American and International Pharmacopoeias, that is to say compositions which are clear and contain no visible particles, but which are also colorless.

“Injectable aqueous solution” is understood to mean solutions of which the solvent is water, which satisfy the conditions of the EP and US Pharmacopoeias.

“Co-polyamino acid consisting of glutamic or aspartic units” is understood to mean noncyclic linear chains of glutamic acid or aspartic acid units bound to one another by peptide bonds, said chains having a C-terminal part corresponding to the carboxylic acid of one extremity, and an N-terminal part corresponding to the amine of the other extremity of the chain.

“Soluble” is understood to mean capable of enabling the preparation of a clear solution which is free of particles at a concentration of less than 60 mg/mL in distilled water at 25° C.

“Alkyl radical” is understood to mean a linear or branched carbon chain which does not comprise a heteroatom.

The co-polyamino acid is a statistical co-polyamino acid in the chain of the glutamic and/or aspartic units.

In the formulas, the * indicate the sites of attachments of the different elements represented.

In an embodiment, the composition according to the invention is characterized in that Hy comprises more than 30 carbon atoms.

In an embodiment, the composition according to the invention is characterized in that Hy comprises from 17 to 30 carbon atoms.

In an embodiment, the composition according to the invention is characterized in that Hy comprises from 19 to 25 carbon atoms

In an embodiment, when p=1, x is from 11 to 25 (11≤x≤25). In particular, when x is from 15 to 16 (x=15 or 16), then r=1 and R is an ether or polyether radical, and, when x is greater than 17 (x≥17), then r=1 and R is an ether or polyether radical.

In an embodiment, when p=2, x is from 9 to 15 (9≤x≤15).

In an embodiment, the composition according to the invention is characterized in that said hydrophobic radicals are chosen from the hydrophobic radicals according to formula I in which p=1, represented by the following formula V:

*(GpR)ᵣ(GpA)ₐGpC formula V

GpR, GpA, GpC, r and a have the definitions given above.

In an embodiment, the composition according to the invention is characterized in that the hydrophobic radical is a radical according to formula V, in which r is equal to 1 (r=1), and a is equal to 0 (a=0).

In an embodiment, the composition according to the invention is characterized in that the hydrophobic radical is a radical according to formula V in which r is equal to 1 (r=1) and a is equal to 1 (a=1).

In an embodiment, the composition according to the invention is characterized in that the hydrophobic radical is a radical according to formula V in which GpR is a radical according to formula II.

In an embodiment, the composition according to the invention is characterized in that the hydrophobic radical is a radical according to formula V in which GpR is a radical according to formula II, in which R is a divalent linear alkyl radical comprising from 2 to 12 carbon atoms.

In an embodiment, the composition according to the invention is characterized in that the hydrophobic radical is a radical according to formula V in which GpR is a radical according to formula H in which R is a divalent alkyl radical comprising from 2 to 6 carbon atoms.

In an embodiment, the composition according to the invention is characterized in that the hydrophobic radical is a radical according to formula V in which GpR is a radical according to formula II in which R is a divalent linear alkyl radical comprising from 2 to 6 carbon atoms.

In an embodiment, the composition according to the invention is characterized in that the hydrophobic radical is a radical according to formula V in which GpR is a radical according to formula II in which R is a divalent alkyl radical comprising from 2 to 4 carbon atoms.

In an embodiment, the composition according to the invention is characterized in that the hydrophobic radical is a radical according to formula V in which GpR is a radical according to formula II in which R is a divalent linear alkyl radical comprising from 2 to 4 carbon atoms.

In an embodiment, the composition according to the invention is characterized in that the hydrophobic radical is a radical according to formula V in which GpR is a radical according to formula II in which R is a divalent alkyl radical comprising 2 carbon atoms.

In an embodiment, the composition according to the invention is characterized in that the hydrophobic radical is a radical according to formula V in which GpR is a radical according to formula II in which R is a divalent alkyl radical comprising 6 carbon atoms.

In an embodiment, the composition according to the invention is characterized in that the hydrophobic radical is a radical according to formula V in which GpR is a radical according to formula II′.

In an embodiment, the composition according to the invention is characterized in that the hydrophobic radical is a radical according to formula V in which GpR is a radical according to formula II′ in which R is a divalent linear alkyl radical comprising from 1 to 11 carbon atoms.

In an embodiment, the composition according to the invention is characterized in that the hydrophobic radical is a radical according to formula V in which GpR is a radical according to formula II′ in which R is a divalent alkyl radical comprising from 1 to 6 carbon atoms.

In an embodiment, the composition according to the invention is characterized in that the hydrophobic radical is a radical according to formula V in which GpR is a radical according to formula II or II′, in which R is a divalent alkyl radical comprising front 2 to 5 carbon atoms and bearing one or more amide functions (—CONH₂).

In an embodiment, the composition according to the invention is characterized in that the hydrophobic radical is a radical according to formula V in which GpR is a radical according to formula II′ or II, in which R is a divalent linear alkyl radical comprising from 2 to 5 carbon atoms and bearing one or more amide functions (—CONH₂).

In an embodiment, the composition according to the invention is characterized in that the hydrophobic radical is a radical according to formula V in which GpR is a radical according to formula II or II′ in which R is a radical chosen from the group consisting of the radicals represented by the formulas below:

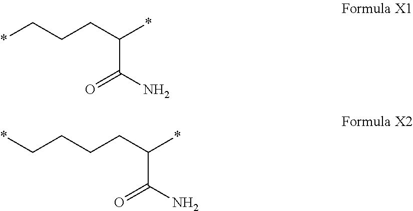 [SMILES: *CCCC(*)C(N)=O ; *CCCCC(*)C(N)=O]

In an embodiment, the composition is characterized in that the hydrophobic radical according to formula V in which GpR is a radical according to formula II or III in which R is a radical according to formula X1.

In an embodiment, the composition is characterized in that the hydrophobic radical according to formula V in which GpR, is a radical according to formula II or II′, in which R is a radical according to formula X2.

In an embodiment, the composition according to the invention is characterized in that the radical R is bound to the co-polyamino acid via an amide function borne by the carbon in delta or epsilon position (or in position 4 or 5 with respect to the amide function (—CONH₂),

In an embodiment, the composition according to the invention is characterized in that the hydrophobic radical is a radical according to formula V in which GpR is a radical according to formula II or II′, in which R is an unsubstituted linear ether or polyether radical comprising from 4 to 14 carbon atoms and from 1 to 5 oxygen atoms.

In an embodiment, the composition according to the invention is characterized in that the hydrophobic radical is a radical according to formula V in which GpR is a radical according to formula II or II′, in which R is an ether radical.

In an embodiment, the composition according to the invention is characterized in that the hydrophobic radical is a radical according to formula V in which GpR is a radical according to formula II or II′, in which R is an ether radical comprising from 4 to 6 carbon atoms.

In an embodiment, the composition according to the invention is characterized in that the hydrophobic radical is a radical according to formula V in which GpR is a radical according to formula II or II′ in which R is an ether radical represented by the formula

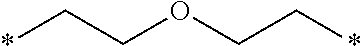 [SMILES: *CCOCC*]

In an embodiment, the composition according to the invention is characterized in that the hydrophobic radical is a radical according to formula V in which GpR is a radical according to formula II or II′, in which R is a polyether radical.

In an embodiment, the composition according to the invention is characterized in that the hydrophobic radical is a radical according to formula V in which GpR is a radical according to formula II or II′, in which R is a linear polyether radical comprising from 6 to 10 carbon atoms and from 2 to 3 oxygen atoms.

In an embodiment, the composition according to the invention is characterized in that the hydrophobic radical according to formula V in which GpR is a radical according to formula II or II′, in which R is a polyether radical chosen from the group consisting of the radicals represented by the formulas below:

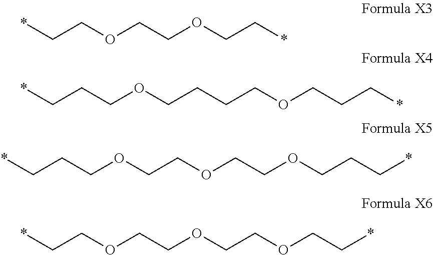 [SMILES: *CCOCCOCC* ; *CCCOCCCCOCCC* ; *CCCOCCOCCOCCC* ; *CCOCCOCCOCC*]

In an embodiment, the composition is characterized in that the hydrophobic radical according to formula V in which GpR is a radical according to formula II or II′, in which R is a radical according to formula X3.

In an embodiment, the composition is characterized in that the hydrophobic radical according to formula V in which GpR is a radical according to formula II or II′, in which R is a radical according to formula X4.

In an embodiment, the composition is characterized in that the hydrophobic radical according to formula V in which GpR is a radical according to formula II or II′, in which R is a radical according to formula X5.

In an embodiment, the composition is characterized in that the hydrophobic radical according to formula V in which GpR is a radical according to formula II or II′, in which R is a radical according to formula X6.

In an embodiment, the composition according to the invention is characterized in that the hydrophobic radical according to formula V in which GpR is a radical in which R is a polyether radical chosen from the group consisting of the radicals represented by the formulas below.

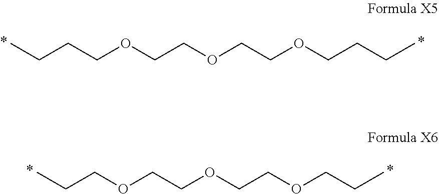

In an embodiment, the composition is characterized in that the hydrophobic radical according to formula V in which GpR is a radical according to formula II in which R is a polyether radical according to formula X5.

In an embodiment, the composition is characterized in that the hydrophobic radical according to formula V in which GpR is a radical according to formula II in which R is a polyether radical according to formula X6.

In an embodiment, the composition according to the invention is characterized in that the hydrophobic radical is a radical according to formula V in which a is equal to 0 (a=0) and r is equal to 0 (r=0).

In an embodiment, the composition according to the invention is characterized in that the hydrophobic radical is a radical according to formula V in which a is equal to 1 (a=1), and A of the the radical GpA according to formula III′ is chosen from the group consisting of the radicals represented by the formulas below:

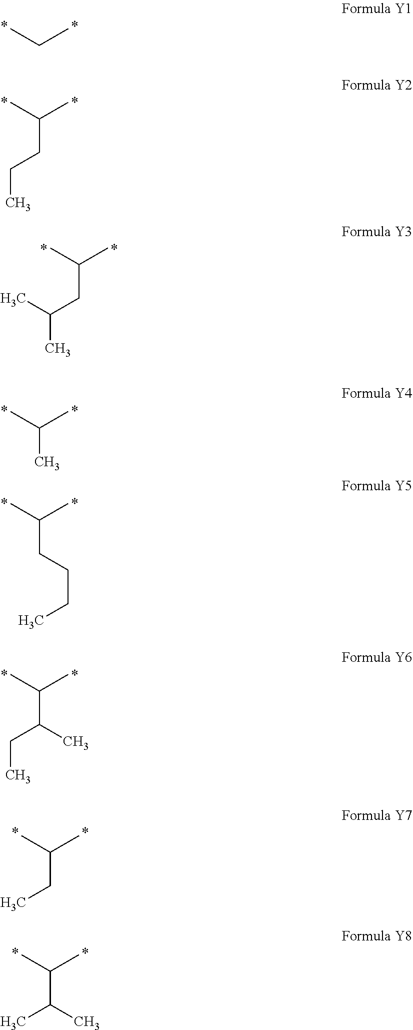

In an embodiment, the composition is characterized in that the hydrophobic radical is a radical according to formula V in which a is equal to 1 (a=1), and A of the radical GpA according to formula III′ is a radical according to formula Y1.

In an embodiment, the composition is characterized in that the hydrophobic radical is a radical according to formula V in which a is equal to 1 (a=1), and A of the radical GpA according to formula III′ is a radical according to formula Y2.

In an embodiment, the composition is characterized in that the hydrophobic radical is a radical according to formula V in which a is equal to 1 (a=1) and A of the radical GpA according to formula III′ is a radical according to formula Y3.

In an embodiment, the composition is characterized in that the hydrophobic radical is a radical according to formula V in which a is equal to 1 (a=1) and A of the radical GpA according to formula III′ is a radical according to formula Y4.

In an embodiment, the composition is characterized in that the hydrophobic radical is a radical according to formula V in which a is equal to 1 (a=1) and A of the radical GpA according to formula III′ is a radical according to formula Y5.

In an embodiment, the composition is characterized in that the hydrophobic radical is a radical according to formula V in which a is equal to 1 (a=1) and A of the radical GpA according to formula III′ is a radical according to formula Y6.

In an embodiment, the composition is characterized in that the hydrophobic radical is a radical according to formula V in which a is equal to 1 (a=1) and A of the radical GpA according to formula III′ is a radical according to formula Y7.

In an embodiment, the composition is characterized in that the hydrophobic radical is a radical according to formula V in which a is equal to 1 (a=1) and A of the radical GpA according to formula III′ is a radical according to formula Y8.

In an embodiment, the composition according to the invention is characterized in that the hydrophobic radical is a radical according to formula V in which the radical GpC according to formula IV is chosen from the group consisting of the radicals according to formula IVa, IVb or IVc represented hereafter:

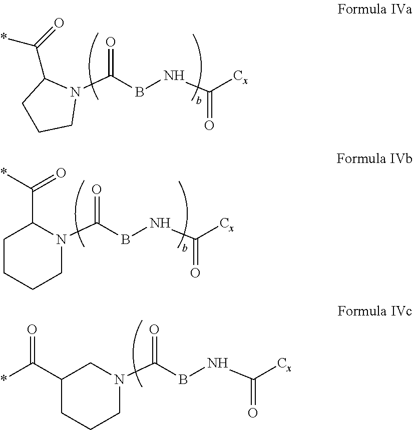

In an embodiment, the composition according to the invention is characterized in that the hydrophobic radical is a radical according to formula V in which the radical GpC is according to formula IVa.

In an embodiment, the composition according to the invention is characterized in that the hydrophobic radical is a radical according to formula V in which the radical GpC according to formula IV is chosen from the group consisting of the radicals according to formula IVa, IVb or IVc in which b is equal to 0, having formulas IVd, IVe and IVf, respectively, represented hereafter:

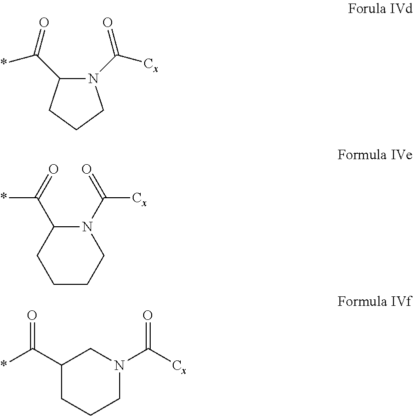

In an embodiment, the composition according to the invention is characterized in that the hydrophobic radical is a radical according to formula V in which the radical GpC corresponds to formula IV or IVa in which b=0, and corresponds to formula IVd.

In an embodiment, the composition according to the invention is characterized in that the hydrophobic radical is a radical according to formula V in which the radical GpC according to formula IV in which b=1 is chosen from the group consisting of the radicals in which B is an amino acid residue chosen from the group consisting of the radicals represented by the formulas below:

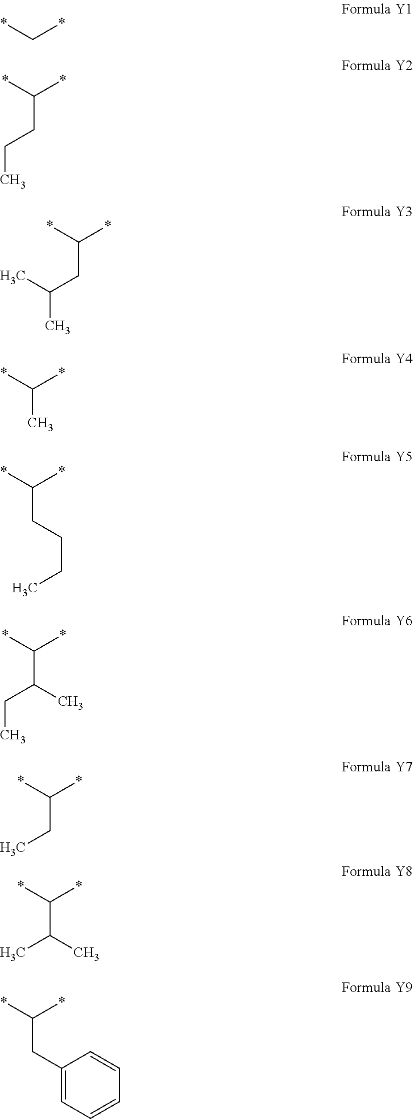

In an embodiment, the composition according to the invention is characterized in that the hydrophobic radical is a radical according to formula V in which the radical GpC according to formula corresponds to formula IV or IVa in which b=1, is chosen from the group consisting of the radicals in which B is an amino acid residue chosen from the group consisting of the radicals represented by the formulas below:

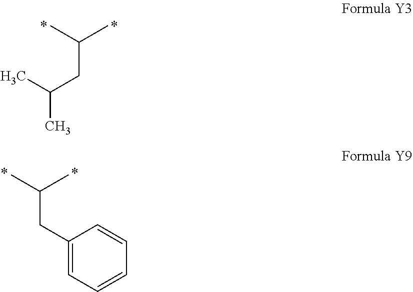

In an embodiment, the composition according to the invention is characterized in that the hydrophobic radical is a radical according to formula V in which the radical GpC according to formula IV is chosen from the group consisting of the radicals in which Cx is chosen from the group consisting of the linear alkyl radicals.

In an embodiment, the composition according to the invention is characterized in that the hydrophobic radical is a radical according to formula V in which the radical GpC according to formula IV is chosen from the group consisting of radicals in which Cx is chosen from the group consisting of the branched alkyl radicals.

In an embodiment, the composition according to the invention is characterized in that the hydrophobic radical is a radical according to formula V in which the radical GpC according to formula IV is chosen from the group consisting of radicals in which Cx is chosen from the group consisting of the radicals comprising from 11 to 14 carbon atoms.

In an embodiment, the composition according to the invention is characterized in that the hydrophobic radical is a radical according to formula V in which the radical GpC according to formula IV is chosen from the group consisting of radicals in which Cx is chosen from the group consisting of the radicals represented by the formulas below:

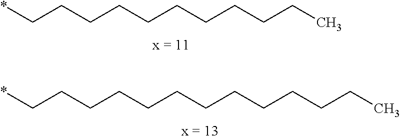 [SMILES: *CCCCCCCCCCC ; *CCCCCCCCCCCCC]

In an embodiment, the composition according to the invention is characterized in that the hydrophobic radical is a radical according to formula V in which the radical GpC according to formula IV is chosen from the group consisting of the radicals in which Cx is chosen from the group consisting of the alkyl radicals comprising from 15 to 16 carbon atoms.

In an embodiment, the composition according to the invention is characterized in that the hydrophobic radical is a radical according to formula V in which the radical GpC according to formula IV is chosen from the group consisting of the radicals in which Cx is chosen from the group consisting of the radicals represented by the formulas below:

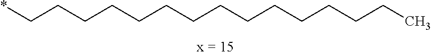 [SMILES: *CCCCCCCCCCCCCCC]

In an embodiment, the composition according to the invention is characterized in that the hydrophobic radical is a radical according to formula V in which the radical GpC according to formula IV is chosen from the group consisting of the radicals in which Cx is chosen from the group consisting of the radicals represented by the formulas below:

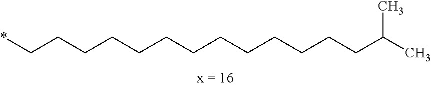 [SMILES: *CCCCCCCCCCCCCC(C)C]

In an embodiment, the composition according to the invention is characterized in that the hydrophobic radical is a radical according to formula V in which the radical GpC according to formula IV is chosen from the group consisting of the radicals in which Cx is chosen from the group consisting of the alkyl radicals comprising from 17 to 25 carbon atoms.

In an embodiment, the composition according to the invention is characterized in that the hydrophobic radical is a radical according to formula V in which the radical GpC according to formula IV is chosen from the group consisting of the radicals in which Cx is chosen from the group consisting of the alkyl radicals comprising from 17 to 18 carbon atoms.

In an embodiment, the composition according to the invention is characterized in that the hydrophobic radical is a radical according to formula V in which the radical GpC according to formula IV is chosen from the group consisting of the radicals in which Cx is chosen from the group consisting of the alkyl radicals represented by the formulas below:

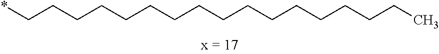 [SMILES: *CCCCCCCCCCCCCCCCC]

In an embodiment, the composition according to the invention is characterized in that the hydrophobic radical is a radical according to formula V in which the radical GpC according to formula IV is chosen from the group consisting of the radicals in which Cx is chosen from the group consisting of the alkyl radicals comprising from 19 to 25 carbon atoms.

In an embodiment, the composition according to the invention is characterized in that the hydrophobic radical is a radical according to formula V in which the radical GpC according to formula IV is chosen from the group consisting of the radicals in which Cx is chosen from the group consisting of the alkyl radicals represented by the formulas below:

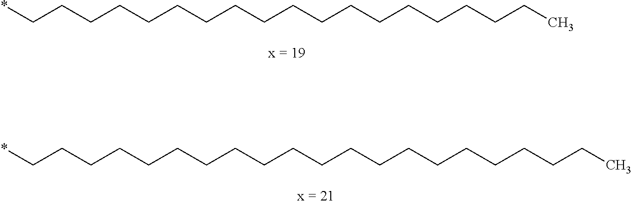 [SMILES: *CCCCCCCCCCCCCCCCCCC ; *CCCCCCCCCCCCCCCCCCCCC]

In an embodiment, the composition according to the invention is characterized in that the hydrophobic radical is a radical according to formula V in which the radical GpC according to formula IV is chosen from the group consisting of the radicals in which Cx is chosen from the group consisting of the alkyl radicals comprising from 18 to 19 carbon atoms

In formulas I and V, the * indicate the sites of attachment of the hydrophobic radicals to co-polyamino acid. The radicals Hy are attached to the co-polyamino acid via amide functions.

In formulas II and II′, the * indicate, from left to right, respectively, the sites of attachment of GpR:
to the co-polyamino acid and
to GpA if a=1, or to GpC if a=0.

In formulas III and III′, the * indicate, from left to right, respectively, the sites of attachment of GpA:
to GpR if r=1, or to the co-polyamino acid if r=0, and
to GpC.

In formula 1V, the * indicates the site of attachment of GpC:
to GpA if a=1, GpR if r=1 and a=0, or to the co-polyamino acid if r=0 and a 0.

All the attachments between the different groups GpR, GpA and GpC are amide functions.

The radicals Hy, GpR, OpA, GpC and D are each independently identical or different from one residue to the other.

When the co-polyamino acid comprises one aspartic unit or several aspartic units, it-they can undergo structural rearrangements.

In embodiments, the composition according to the invention is characterized in that the co-polyamino acid which bears carboxylate charges and hydrophobic radicals is chosen from the co-polyamino acids of the following formula VII:

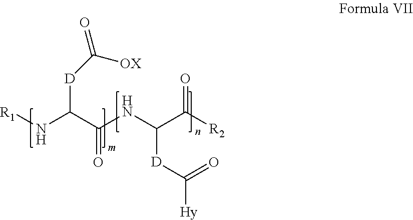

in which,
D represents, independently, either a —CH₂— group (aspartic unit) or a —CH₂-CH₂— group (glutamic unit),
Hy is a hydrophobic radical chosen from the hydrophobic radicals according to formula I or V, such as hydrophobic radicals according to formula I or V in which r=1 and GpR is a radical according to formula II,
R₁ is a hydrophobic radical chosen from the hydrophobic radicals according to formula or V such as hydrophobic radicals according to formula I or V in which r=0 or r=1 and GpR, is a radical according to formula II′, or a radical chosen from the group consisting of H, a C2 to C10 linear acyl group; a C3 to C10 branched acyl group such as a C4 to C10 branched acyl group, benzyl, a terminal “amino acid” unit and a pyroglutamate,
R₂ is a hydrophobic radical chosen from the hydrophobic radicals according to formula I or V VI in which r=1 and GpR is a radical according to formula II, a —NR′R″ radical R′ and R″ which are identical or different being chosen from the group consisting of H, the C2 to C 10 linear or branched or cyclic alkyls, benzyl, and said alkyl R′ and R″ may form one or more saturated, unsaturated and/or aromatic carbon rings and/or may comprise heteroatoms chosen from the group consisting of O, N and S;
* X represents a cationic entity chosen from the group comprising the alkaline cations;
n+m represents the degree of polymerization DP of the co-polyamino acid, that is to say the average number of monomer units per co-polyamino acid chain, and 5≤n+m≤250.

In an embodiment, the composition according to the invention is characterized in that, when the co-polyamino acid comprises aspartate units, then the co-polyamino acid can, in addition, comprise monomer units according to formula VIII and/or VIII′:

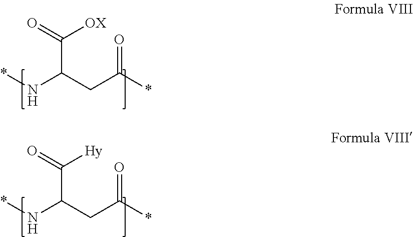

“Co-polyamino acid with statistical grafting” is used to denote a co-polyamino acid bearing carboxylate charges and at least one hydrophobic radical, a co-polyamino acid according to formula VIIa.

In an embodiment, the composition according to the invention is characterized in that the co-polyamino acid bearing carboxylate charges and hydrophobic radicals is chosen from the co-polyamino acids according to formulas VII, in which R₁═R′₁ and R₂=R₂, of following formula VIIa:

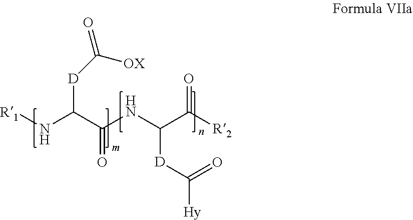

in which
m, n, X, D and Hy have the definitions given above,
R′₁ is a radical chosen from the group consisting of H, a C2 to C10 linear acyl group, a C3 to C10 branched acyl group such as a C4 to C10 branched acyl group, benzyl, a terminal “amino acid” unit and a pyroglutamate,
R′₂ is a —NR′R″ radical, R′ and R″ which are identical or different being chosen from the group consisting of H, the C2 to C10 linear or branched or cyclic alkyls, benzyl, and said alkyl R′ and R″ may form together one or more saturated, unsaturated and/or aromatic carbon rings and/or may comprise heteroatoms chosen from the group consisting of O, N and S.

In an embodiment, the composition according to the invention is characterized in that the co-polyamino acid bearing carboxylate charges and hydrophobic radicals is chosen from the co-polyamino acids according to formulas VIIa, in which Hy is a radical according to formula V.

In an embodiment, the composition according to the invention is characterized in that the co-polyamino acid bearing carboxylate charges and hydrophobic radicals is chosen from the co-polyamino acids according to formula VIIa, in which Hy is a radical according to formula V in which r=1.

In an embodiment, the composition according to the invention is characterized in that the co-polyamino acid bearing carboxylate charges and hydrophobic radicals is chosen from the co-polyamino acids according to formulas VIIa, in which Hy is a radical according to formula V, in which r=1, and for GpC, b=0.

In an embodiment, the composition according to the invention is characterized in that the co-polyamino acid bearing carboxylate charges and hydrophobic radicals is chosen from the co-polyamino acids according to formula VIIa, in which Hy is a radical according to formula V, and in which GpC is a radical according to formula IVd.

In an embodiment, the composition according to the invention is characterized in that the co-polyamino acid bearing carboxylate charges and hydrophobic radicals is chosen from the co-polyamino acids according to formula VIIa, in which Hy is a radical according to formula V, and in which GpC is a radical according to formula IVd and r=1.

In an embodiment, the composition according to the invention is characterized in that the co-polyamino acid bearing carboxylate charges and hydrophobic radicals is chosen from the co-polyamino acids according to formula VIIa in which Hyd is according to formula V in which r=1, GpR is according to formula II, a=0 and Gpc is according to formula IVd.

In an embodiment, the composition according to the invention is characterized in that the co-polyamino acid bearing carboxylate charges arid hydrophobic radicals is chosen from the co-polyamino acids according to formula VIIa in which Hyd is according to formula V in which r=1, GpR is according to formula II, a=0 and Gpc is according to formula IVd, where x=11.

In an embodiment, the composition according to the invention is characterized in that the co-polyamino acid bearing carboxylate charges and hydrophobic radicals is chosen from the co-polyamino acids according to formula VIIa in which Hyd is according to formula V in which r=1, GpR is according to formula II, a=0 and Gpc is according to formula IVd, where x=13.

In an embodiment, the composition according to the invention is characterized in that the co-polyamino acid bearing carboxylate charges and hydrophobic radicals is chosen from the co-polyamino acids according to formula VIIa in which Hyd is according to formula V in which r=1, GpR is according to formula II, a=0 and Gpc is according to formula IVd where x=15.

In an embodiment, the composition according to the invention is characterized in that the co-polyamino acid bearing carboxylate charges and hydrophobic radicals is chosen from the co-polyamino acids according to formula VIIa in which Hyd is according to formula V in which r=1, GpR is according to formula II, a=0 and Gpc is according to formula IVd where x=17.

“Co-polyamino acid with defined grafting” denotes a co-polyamino acid bearing carboxylate charges and at least one hydrophobic radical, a co-polyamino acid according to formula VIIb.

In an embodiment, the composition according to the invention is characterized in that the co-polyamino acid hearing carboxylate charges and hydrophobic radicals is chosen from the co-polyamino acids according to formula VII in which n=0 of following formula VIIb:

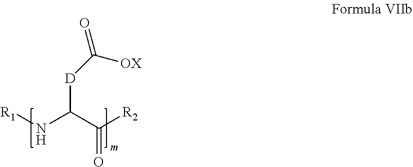

in which m, X, D, R₁ and R₂ have the definitions given above and at least R₁ or R₂ is a hydrophobic radical according to formula I or V I.
In an embodiment, the composition according to the invention is characterized in that the co-polyamino acid bearing carboxylate charges and hydrophobic radicals is chosen from the co-polyamino acids according to formula VII in which n=0 according to formula VIIb and R₁ or R₂ is a hydrophobic radical according to formula I or V.

In an embodiment, the composition according to the invention is characterized in that the co-polyamino acid bearing carboxylate charges and hydrophobic radicals is chosen from the co-polyamino acids according to formula VIIb, in which R₁=R′₁, according to formula VIIb′:

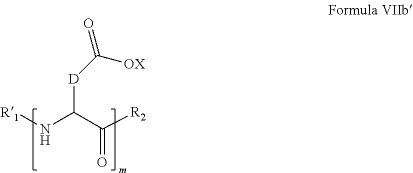

in which in, X, D, R′₁ and R₂ have the definitions given above and R₂ is a hydrophobic radical according to formula I or V.

In an embodiment, the composition according to the invention is characterized in that the co-polyamino acid bearing carboxylate charges and hydrophobic radicals is chosen from the co-polyamino acids according to formula VIIb, in which R₂=R′₂; according to formula VIIb″,

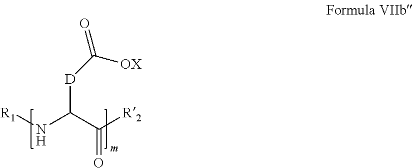

in which m, X, D, R₁ and R′₂, have the meanings given above and R₁ is a hydrophobic radical according to formula I or V.

In an embodiment, the composition according to the invention is characterized in that the co-polyamino acid bearing carboxylate charges and hydrophobic radicals is chosen from the co-polyamino acids according to formula VIIb or VIIb″ in which R₁ is a hydrophobic radical according to formula I or V in which r=0 or r=1 and GpR is according to formula II′.

In an embodiment, the composition according to the invention is characterized in that the co-polyamino acid bearing carboxylate charges and hydrophobic radicals is chosen from the co-polyamino acids according to formula VIIb or VIIb″ in which R′₁ is a hydrophobic radical according to formula V and GpR is according to formula II′.

In an embodiment, the composition according to the invention is characterized in that the co-polyamino acid bearing carboxylate charges and hydrophobic radicals is chosen from the co-polyamino acids according to formula VIIb or VIIb″ in which R₁ is a hydrophobic radical according to formula V and GpR is according to formula II′ and GpC is according to formula IVa.

In an embodiment, the composition according to the invention is characterized in that the co-polyamino acid bearing carboxylate charges and hydrophobic radicals is chosen from the co-polyamino acids according to formula VIIb or VIIb″ in which R₁ is a hydrophobic radical according to formula V and GpR is according to formula II′ and GpC is according to formula IVd.

In an embodiment, the composition according to the invention is characterized in that the co-polyamino acid bearing carboxylate charges and hydrophobic radicals is chosen from the co-polyamino acids according to formula VIIb or VIIb′ in which R₂ is a hydrophobic radical according to formula I or V in which r=1 and GpR is according to formula II.

In an embodiment, the composition according to the invention is characterized in that the co-polyamino acid bearing carboxylate charges and hydrophobic radicals is chosen from the co-polyamino acids according to formula VIIb′ in which R₂ is a hydrophobic radical according to formula V in which r=1 and GpR is according to formula II and a=0.

In an embodiment, the composition according to the invention is characterized in that the co-polyamino acid bearing carboxylate charges and hydrophobic radicals is chosen from the co-polyamino acids according to formula VIIb′ in which R₂ is a hydrophobic radical according to formula V in which r=1, GpR is according to formula II and a=0 and GpC is according to formula IVa or IVc.

In an embodiment, the composition according to the invention is characterized in that the co-polyamino acid bearing carboxylate charges and hydrophobic radicals is chosen from the co-polyamino acids according to formula VIIb′ in which R₂ is a hydrophobic radical according to formula V in which r=1, GpR is according to formula II and a=0 and GpC is according to formula IVa.

In an embodiment, the composition according to the invention is characterized in that the co-polyamino acid bearing carboxylate charges and hydrophobic radicals is chosen from the co-polyamino acids according to formula VIIb′ in which R₂ is a hydrophobic radical according to formula V in which r=1, GpR is according to formula II and a=0 and GpC is according to formula IVc.

In an embodiment, the composition according to the invention is characterized in that the co-polyamino acid hearing carboxylate charges and hydrophobic radicals is chosen from the co-polyamino acids according to formula in which R₂ is a hydrophobic radical according to formula V in which r=1, GpR is according to formula II and a=0 and GpC is according to formula IVd or IVf. In an embodiment, the composition according to the invention is characterized in that the co-polyamino acid bearing carboxylate charges and hydrophobic radicals is chosen from the co-polyamino acids according to formula VIIb′ in which R₂ is a hydrophobic radical according to formula V in which r=1, GpR is according to formula II and a=0 and GpC is according to formula IVd.

In an embodiment, the composition according to the invention is characterized in that the co-polyamino acid bearing carboxylate charges and hydrophobic radicals is chosen from the co-polyamino acids according to formula VIIb′ in which R₂ is a hydrophobic radical according to formula V in which r=1, GpR is according to formula II and a=0 and GpC is according to formula IVf.

In an embodiment, the composition according to the invention is characterized in that the co-polyamino acid bearing carboxylate charges and hydrophobic radicals is chosen from the co-polyamino acids according to formulas VIIb′ in which R₂ is a hydrophobic radical according to formula V in which r=1 and GpR is according to formula II and a=1.

In an embodiment, the composition according to the invention is characterized in that the co-polyamino acid bearing carboxylate charges and hydrophobic radicals is chosen from the co-polyamino acids according to formulas VIIb′ in which R₂ is a hydrophobic radical according to formula V in which r=1, GpR is according to formula II, a=1 and GpC is according to formula IVa or IVd.

In an embodiment, the composition according to the invention is characterized in that the co-polyamino acid bearing carboxylate charges and hydrophobic radicals is chosen from the co-polyamino acids according to formulas ‘alb’ in which R₂ is a hydrophobic radical according to formula V in which r=1, GpR is according to formula II, a=1 and GpC is according to formula IVd.

In an embodiment, the composition according to the invention is characterized in that the co-polyamino acid bearing carboxylase charges and hydrophobic radicals is chosen from the co-polyamino acids according to formulas VIIb′ in which R₂ is a hydrophobic radical according to formula V in which r=1, GpR is according to formula II , a=1 and GpC is according to formula IVd, with x=11.

In an embodiment, the composition according to the invention is characterized in that the co-polyamino acid bearing carboxylate charges and hydrophobic radicals is chosen from the co-polyamino acids according to formulas VIIb′ in which R₂ is a hydrophobic radical according to formula V in which r=1, GpR is according to formula II, a=1 and GpC is according to formula IVd, with x=13.

In an embodiment, the composition according to the invention is characterized in that the co-polyamino acid bearing carboxylate charges and hydrophobic radicals is chosen from the co-polyamino acids according to formulas VIIb′ in which R₂ is a hydrophobic radical according to formula V in which r=1, GpR is according to formula II, a=1 and GpC is according to formula IVd with x=15.

In an embodiment, the composition according to the invention is characterized in that the co-polyamino acid bearing carboxylate charges and hydrophobic radicals is chosen from the co-polyamino acids according to formulas VIIb′ in which R₂ is a hydrophobic radical according to formula V in which r=1, GpR is according to formula II, a=0 and GpC is according to formula IVd.

In an embodiment, the composition is characterized in that the co-polyamino acid bearing carboxylate charges and hydrophobic radicals is chosen from the co-polyamino acids according to the following formula XX:

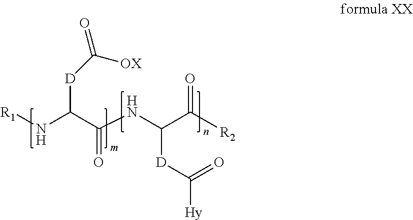

in which,
D represents, independently, either a —CH₂— group (aspartic unit) or a —CH₂-CH₂— group (glutamic unit),
Hy is a hydrophobic radical chosen from the hydrophobic radicals according to formula I or V, in which r=1 and GpR is a radical according to formula II,
R₁ is a hydrophobic radical chosen from the hydrophobic radicals according to formula I or V in which r=0 or r=1 and GpR is a radical according to formula II′, or a radical chosen from the group consisting of H, a C2 to C10 linear acyl group, a C4 to C10 branched acyl group, benzyl, a terminal “amino acid” unit and a pyroglutamate,
R₂ is a hydrophobic radical chosen from the hydrophobic radicals according to formula I or V in which r=1 and GpR is a radical according to formula II, or a —NR′R″ radical, R′ and R″, which are identical or different, being chosen from the group consisting of H, the C2 to C10 linear or branched or cyclic alkyls, benzyl and said alkyl R′ and R″ may form together one or more saturated, unsaturated and/or aromatic carbon rings and/or may comprise heteroatoms, chosen from the group consisting of O, N and S,
at least one of the R₁ or R₂ is a hydrophobic radical as defined above,
a X represents H or a cationic entity chosen from the group comprising the metallic cations;
n+m represents the degree of polymerization DP of the co-polyamino acid, that is to say the average number of monomer units per co-polyamino acid chain and 5≤n+m≤250.

In an embodiment, the composition according to the invention is characterized in that R₁ is a radical chosen from the group consisting of a C₂ to C₁₀ linear acyl group, a C₄ to C₁₀ branched acyl group, benzyl, a terminal “amino acid” group and a pyroglutamate.

In an embodiment, the composition according to the invention is characterized in that R₁ is a radical chosen from the group consisting of a C₂ to C₁₀ linear acyl group or a C₄ to C₁₀ branched acyl group.

In an embodiment, the composition according to the invention is characterized in that the co-polyamino acid bearing carboxylate charges and hydrophobic radicals is chosen from the co-polyamino acids according to formula VII, VIIa, VIIb, VIIb′, VIIb″ or XX in which the co-polyamino acid is chosen from the co-polyamino acids in which the group D is a —CH₂— group (aspartic unit).

In an embodiment, the composition according to the invention is characterized in that the co-polyamino acid bearing carboxylate charges and hydrophobic radicals is chosen from the co-polyamino acids according to formula VII, VIIa, VIIb, VIIb′, VIIb″ or XX in which the co-polyamino acid is chosen from the co-polyamino acids in which the group D is a —CH₂-CH₂— group (glutamic unit).

The ratio of hydrophobic radical to basal insulin is defined to be the ratio of their respective molar concentrations: [Hy]/[basal insulin] (mol/mol) until obtaining the expected performances, namely the solubilization of basal insulin at a pH from 6.0 to 8.0, the precipitation of the basal insulin, and the stability of the compositions according to the invention.

The minimum value of the ratio of hydrophobic radical to basal insulin [Hy]/[basal insulin] measured is the value at which the basal insulin is solubilized, since the solubilization is the minimum effect to be obtained; this solubilization is a condition for all the other technical effects which can be only observed if the basal insulin is solubilized at a uH from 6.0 to 8.0.

In the compositions according to the invention, the ratio of hydrophobic radical over basal insulin [Hy]/[basal insulin] can be greater than the minimum value determined by the solubilization limit.

In an embodiment, the ratio of hydrophobic radical to basal insulin [Hy]/[basal insulin]≤2.

In an embodiment, the ratio of hydrophobic radical to basal insulin [Hy]/[basal insulin]≤1.75.

In an embodiment, the ratio of hydrophobic radical to basal insulin [Hy]/[basal insulin]≤1.5.

In an embodiment, the ratio of hydrophobic radical to basal insulin [Hy]/[basal insulin]=1.25.

In an embodiment, the ratio of hydrophobic radical to basal insulin [Hy]/[basal insulin]≤1.00.

In an embodiment, the ratio of hydrophobic radical to basal insulin [Hy]/[basal insulin]≤0.75.

In an embodiment, the ratio of hydrophobic radical to basal insulin [Hy]/[basal insulin]≤0.5.

In an embodiment, the ratio of hydrophobic radical to basal insulin [Hy]/[basal insulin]≤0.25.

In an embodiment, the composition according to the invention is characterized in that the ratio i between the number of hydrophobic radicals and the number of glutamic or aspartic units is from 0.007 to 0.3.

In an embodiment, the composition according to the invention is characterized in that the ratio i between the number of hydrophobic radicals and the number of glutamic or aspartic units is from 0.01 to 0.3.

In an embodiment, the composition according to the invention is characterized in that the ratio i between the number of hydrophobic radicals and the number of glutamic or aspartic units is from 0.02 to 0.2.

In an embodiment, the composition according to the invention is characterized in that the hydrophobic radical corresponds to formula V, and the ratio i between the number of hydrophobic radicals and the number of glutamic or aspartic units is from 0.007 to 0.15.

In an embodiment, the composition according to the invention is characterized in that the hydrophobic radical corresponds to formula V, and the ratio i between the number of hydrophobic radicals and the number of glutamic or aspartic units is from 0.01 to 0.1.

In an embodiment, the composition according to the invention is characterized in that the hydrophobic radical corresponds to formula V, and the ratio i between the number of hydrophobic radicals and the number of glutamic or aspartic units is from 0.02 to 0.08.

In an embodiment, the composition according to the invention is characterized in that the hydrophobic radical corresponds to formula V in which the radical Cx comprises from 9 to 10 carbon atoms, and the ratio i between the number of hydrophobic radicals and the number of glutamic or aspartic units is from 0.03 to 0.15.

In an embodiment, the composition according to the invention is characterized in that the hydrophobic radical corresponds to formula V in which the radical Cx comprises from 11 to 12 carbon atoms, and the ratio i between the number of hydrophobic radicals and the number of glutamic, or aspartic units is from 0.015 to 0.1.

In an embodiment, the composition according to the invention is characterized in that the hydrophobic radical corresponds to formula V in which the radical Cx comprises from 11 to 12 carbon atoms, and the ratio i between the number of hydrophobic radicals and the number of glutamic or aspartic units is from 0.02 to 0.08.

In an embodiment, the composition according to the invention is characterized in that the hydrophobic radical corresponds to formula V in which the radical Cx comprises from 13 to 1.5 carbon atoms, and the ratio i between the number of hydrophobic radicals and the number of glutamic or aspartic units is from 0.01 to 0.1.

In an embodiment, the composition according to the invention is characterized in that the hydrophobic radical corresponds to formula V in which the radical Cx comprises from 13 to 15 carbon atoms, and the ratio i between the number of hydrophobic radicals and the number of glutamic or aspartic units is from 0.01 to 0.06.

In an embodiment, the composition according to the invention is characterized in that the hydrophobic radical corresponds to formula V, and the ratio i between the number of hydrophobic radicals and the number Of glutamic or aspartic units is from 0.007 to 0.3.

In an embodiment, the composition according to the invention is characterized in that the hydrophobic radical corresponds to formula V, and the ratio i between the number of hydrophobic radicals and the number of glutamic, or aspartic units is from 0.01 to 0.3.

In an embodiment, the composition according to the invention is characterized in that the hydrophobic radical corresponds to formula V, and the ratio i between the number of hydrophobic radicals and the number of glutamic or aspartic units is from 0.015 to 0.2.

In an embodiment, the composition according to the invention is characterized in that the hydrophobic radical corresponds to formula V in which the radical Cx comprises from 11 to 14 carbon atoms, and the ratio i between the number of hydrophobic radicals and the number of glutamic or aspartic units is from 0.1 to 0.2.

In an embodiment, the composition according to the invention is characterized in that the hydrophobic radical corresponds to formula V in which the radical Cx comprises from 15 to 16 carbon atoms, and the ratio i between the number of hydrophobic radicals and the number of glutamic or aspartic units is from 0.04 to 0.15.

In an embodiment, the composition according to the invention is characterized in that the hydrophobic radical corresponds to formula V in which the radical Cx comprises from 17 to 18 carbon atoms, and the ratio i between the number of hydrophobic radicals and the number of glutamic or aspartic units is from 0.02 to 0.06.

In an embodiment, the composition according to the invention is characterized in that the hydrophobic radical corresponds to formula V in which the radical Cx comprises from 19 to 25 carbon atoms, and the ratio i between the number of hydrophobic radicals and the number of glutamic or aspartic units is from 0.01 to 0.06.

In an embodiment, the composition according to the invention is characterized in that the hydrophobic radical corresponds to formula V in which the radical Cx comprises from 19 to 25 carbon atoms, and the ratio i between the number of hydrophobic radicals and the number of glutamic or aspartic units is from 0.01 to 0.05.

In an embodiment, the composition according to the invention is characterized in that n+m is from 10 to 200.

In an embodiment, the composition according to the invention is characterized in that n+m is from 15 to 150.

In an embodiment, the composition according to the invention is characterized in that n+m is from 15 to 100.

In an embodiment, the composition according to the invention is characterized in that n+m is from 15 to 80.

In an embodiment, the composition according to the invention is characterized in that n+m is from 15 to 65.

In an embodiment, the composition according to the invention is characterized in that n+m is from 20 to 60.

In an embodiment, the composition according to the invention is characterized in that n+m is from 20 to 50.

In an embodiment, the composition according to the invention is characterized in that n+m is from 20 to 40.

In an embodiment, the at least one hydrophobic radical according to formula I is chosen from the radicals according to formula I in which r=1, a=0, p=1, GpR corresponds to formula II in which R is

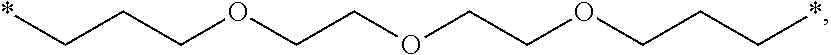

GpC corresponds to formula IVd in which x=15 and Cx is

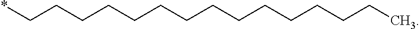 [SMILES: *CCCCCCCCCCCCCCC]

The values of the degree of polymerization DP and of ratio i are estimated by ¹H NMR in D₂O by comparing the integration of the signals resulting from the hydrophobic groups with that of the signals resulting from the main chain of the co-polyamino acid.

In an embodiment, the co-polyamino acid bearing carboxylate charges and hydrophobic radicals is a co-polyamino acid according to formula VII or VIIa, in which DP=25/−5, i=0.07+/−0.02, and the at least one hydrophobic radical according to formula I is chosen from the radicals according to formula I in, which r=1, a=0, p=1, GpR corresponds to formula II in which R is

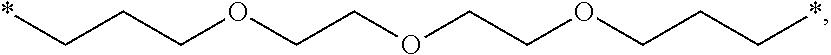

GpC corresponds to formula IVd in which x=15 and Cx is

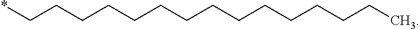 [SMILES: *CCCCCCCCCCCCCCC]

The invention also relates to said co-polyamino acids bearing carboxylate charges and hydrophobic radicals according to formula I or V.

In an embodiment, the invention also relates to the precursors of said. hydrophobic radicals according to formulas I′, V′ and VI′:

H(GpR)ᵣ(GpA)ₐ(GpC)ₚ formula I′

H(GpR)ᵣ(GpA)ₐGpc formula V′

H(GpR)ᵣ(GpA)(GpC)₂ formula VI′

GpR, GpA, Gp r, a, p have the meanings given above.

The invention also relates to a method for preparing stable injectable compositions.

In an embodiment, the composition according to the invention is characterized in that the co-polyamino acid is obtained from a polyamino acid obtained by polymerization.

In an embodiment, the composition according to the invention is characterized in that the co-polyamino acid is obtained from a polyamino acid obtained by ring-opening polymerization of a glutamic acid N-carboxyanhydride derivative or an aspartic acid N-carboxyanhydride derivative.

In an embodiment, the composition according to the invention is characterized in that the co-polyamino acid is obtained from a polyamino acid obtained by polymerization of a glutamic acid N-carboxyanhydride derivative or of an aspartic acid N-carboxyanhydride derivative described in the review article Adv. Polym. Sci. 2006, 202, 1-18 (Deming, T. J.).

In an embodiment, the composition according to the invention is characterized in that the co-polyamino acid is obtained from a polyamino acid obtained by polymerization of a glutamic acid N-carboxyanhydride derivative.

In an embodiment, the composition according to the invention is characterized in that the co-polyamino acid is obtained from a polyamino acid obtained by polymerization of a glutamic acid N-carboxyanhydride derivative chosen from the group consisting of methyl polyglutamate N-carboxyanhydride (GluOMe-NCA), benzyl polyglutamate N-carboxyanhydride (GluOBzl-NCA), and t-butyl polyglutamate N-carboxyanhydride (GluOtBu-NCA).

In an embodiment, the glutamic acid N-carboxyanhydride derivative is methyl poly-L-glutamate N-carboxyanhydride (L-GluOMe-NCA).

In an embodiment, the glutamic acid N-carboxyanhydride derivative is benzyl poly-L-glutamate N-carboxyanhydride (L-GluOBzl-NCA).

In an embodiment, the composition according to the invention is characterized in that the co-polyamino acid is obtained from a polyamino acid obtained by polymerization of a glutamic acid N-carboxyanhydride derivative or of an aspartic acid N-carboxyanhydride derivative, using an organometallic complex of a transition Metal as initiator as described in the publication Nature 1997, 390, 386-389 (Deming, T. J.).

In an embodiment, the composition according to the invention is characterized in that the co-polyamino acid is obtained from a polyamino acid obtained by polymerization of a glutamic acid N-carboxyanhydride derivative or of an aspartic acid N-carboxyanhydride derivative, using ammonia or a primary amine as initiator as described in the patent FR 2,801,226 (Touraud, F.; et al.) and the references cited in this patent.

In an embodiment, the composition according to the invention is characterized in that the co-polyamino acid is obtained from a polyamino acid obtained by polymerization of a glutamic acid N-carboxyanhydride derivative or of an aspartic acid N-carboxyanhydride derivative, using hexamethyldisilazane as initiator as described in the publication J. Am. Chem. Soc, 2007, 129, 14114-14115 (Lu H.; et al.) or a silylated amine as described in the publication J. Am. Chem. Soc. 2008, 130, 12562-12563 (Lu H.; et al.).

In an embodiment, the composition according to the invention is characterized in that the method for synthesizing the polyamino acid obtained by polymerization of a glutamic acid N-carboxyanhydride derivative or an aspartic acid N-carboxyanhydride derivative from which the co-polyamino acid is obtained comprises a step of hydrolysis of the ester functions.

In an embodiment, this step of hydrolysis of the ester functions can consist of a hydrolysis in an acidic medium or a hydrolysis in a basic medium or it can be carried out by hydrogenation.

In an embodiment, this step of hydrolysis of the ester groups is a hydrolysis in an acidic medium.

In an embodiment, this step of hydrolysis of the ester groups is carried out by hydrogenation.

In an embodiment, the composition according to the invention is characterized in that the co-polyamino acid is obtained from a polyamino acid obtained by depolymerization of a polyamino acid of higher molecular weight.

In an embodiment, the composition according to the invention is characterized in that the co-polyamino acid is obtained from a polyamino acid obtained by enzymatic depolymerization of a polyamino acid of higher molecular weight.

In an embodiment, the composition according to the invention is characterized in that the co-polyamino acid is obtained from a polyamino acid obtained by chemical depolymerization of a polyamino acid of higher molecular weight.

In an embodiment, the composition according to the invention is characterized in that the co-polyamino acid is obtained from a polyamino acid obtained by enzymatic and chemical depolymerization of a polyamino acid of higher molecular weight.

In an embodiment, the composition according to the invention is characterized in that the co-polyamino acid is obtained from a polyamino acid obtained by depolymerization of a polyamino acid of higher molecular weight chosen from the group consisting of sodium polyglutamate and sodium polyaspartate.

In an embodiment, the composition according to the invention is characterized in that the co-polyamino acid is obtained from a polyamino acid obtained by depolymerization of a sodium polyglutamate of higher molecular weight.

In an embodiment, the composition according to the invention is characterized in that the co-polyamino acid is obtained from a polyamino acid obtained by depolymerization of a sodium polyaspartate of higher molecular weight.

In an embodiment, the composition according to the invention is characterized in that the co-polyamino acid is obtained by grafting a hydrophobic group onto a poly-L-glutamic acid or poly-L-aspartic acid using the methods for forming amide bonds, which are well known to the person skilled in the art.

In an embodiment, the composition according to the invention is characterized in that the co-polyamino acid is obtained by grafting of a hydrophobic group onto a poly-L-glutamic acid or poly-L-aspartic acid using the methods for forming amide bonds, used in peptide synthesis.

In an embodiment, the composition according to the invention is characterized in that the co-polyamino acid is obtained by grafting of a hydrophobic group onto a poly-L-glutamic acid or poly-L-aspartic acid as described in the patent FR 2,840,614 (Chan, Y. P.; et al,).

Below, the units used for the insulins are those recommended by the pharmacopoeias; the associated correspondences in mg/mL are given in the table below:

| | | |
|---|---|---|
| Insulin | EP Pharmacopoeia EP 8.0 | US Pharmacopoeia - USP38 (2015) |
| | (2014) | |
| Aspart | 1 U = 0.0350 mg of insulin | 1 USP = 0.0350 lug of insulin |
| | aspart | aspart |
| Lispro | 1 U = 0.0347 mg of insulin | 1 USP = 0.0347 mg of insulin |
| | lispro | lispro |
| Human | 1 IU = 0.0347 mg of human | 1 USP = 0.0347 mg of human |
| | insulin | insulin |
| Glargine | 1 U = 0.0364 mg of insulin | 1 USP = 0.0364 mg of insulin |
| | glargine | glargine |
| Pork | 1 IU = 0.0345 mg of pork | 1 USP = 0.0345 mg of pork insulin |
| | insulin | |
| Beef | 1 IU = 0.0342 mg of beef | 1 USP = 0.0342 mg of beef insulin |
| | insulin | |

A basal insulin of which the isoelectric point is from 5.8 to 8.5 is understood to mean an insulin which is insoluble at pH 7 and the duration of action of which is from 8 to 24 hours or more in the standard diabetes models.

These basal insulins whose isoelectric point is from 5.8 to 8.5 are recombinant insulins of which the primary structure has been modified primarily by the introduction of alkaline amino acids such as arginine or lysine. They are described, for example, in the following patents, patent applications or publications WO 2003/053339, WO 7004/096854, U.S. Pat. No. 5,656,722 and U.S. Pat. No.6,100,376 the content of which is incorporated by reference.

In an embodiment, the basal insulin of which the isoelectric point is from 5.8 to 8.5 is insulin glargine. Insulin glargine is marketed under the trade name of Lanais® (100 U/mL) or Toujeo® (300 U/mL) by SANOFI.

In an embodiment, the basal insulin of which the isoelectric point is from 5.8 to 8.5 is a biosimilar insulin glargine.

A biosimilar insulin glargine is in the process of being marketed under the trade name of Abasaglar® or Basaglar® by ELI LILLY.

In an embodiment, the compositions according to the invention comprise from 40 to 500 U/mL of basal insulin of which the isoelectric point is from 5.8 to 8.5.

In an embodiment, the compositions according to the invention comprise 40 U/mL of a basal insulin of which the isoelectric point is from 5.8 to 8.5.

In an embodiment, the compositions according to the invention comprise 100 U/mL (or approximately 3.6 mg/mL) of basal insulin of which the isoelectric point is from 5.8 to 8.5.

In an embodiment, the compositions according to the invention comprise 150 U/mL of basal insulin of which the isoelectric point is from 5.8 to 8.5.

In an embodiment, the compositions according to the invention comprise 200 U/mL of basal insulin of which the isoelectric point is from 5.8 to 8.5.

In an embodiment, the compositions according to the invention comprise 225 U/mL, of basal insulin of which the isoelectric point is from 5.8 to 8.5.

In an embodiment, the compositions according to the invention comprise 250 U/mL of basal insulin of which the isoelectric point is from 5.8 to 8.5.

In an embodiment, the compositions according to the invention comprise 300 U/mL of basal insulin of which the isoelectric point is from 5.8 to 8.5.

In an embodiment, the compositions according to the invention comprise 400 U/mL of basal insulin of which the isoelectric point is from 5.8 to 8.5.

In an embodiment, the compositions according to the invention comprise 500 U/mL of basal insulins of which the isoelectric point is from 5.8 to 8.5.

In an embodiment, the mass ratio between the basal insulin of which the isoelectric point is from 5.8 to 8.5 and the co-polyamino acid, or co-polyamino acid/basal insulin, is from 0.2 to 8.

In an embodiment, the mass ratio is from 0.2 to 6.

In an embodiment, the mass ratio is from 0.2 to 5.

In an embodiment, the mass ratio is from 0.2 to 4.

In an embodiment, the mass ratio is from 0.2 to 3.

In an embodiment, the mass ratio is from 0.2 to 2.

In an embodiment, the mass ratio is from 0.2 to 1.

In an embodiment, the concentration of co-polyamino acid bearing carboxylate charges and hydrophobic radicals is at most 60 mg/mL.

In an embodiment, the concentration of co-polyamino acid bearing carboxylate charges and hydrophobic radicals is at most 40 mg/mL.

In an embodiment, the concentration of co-polyamino acid bearing carboxylate charges and hydrophobic radicals is at most 20 mg/mL.

In an embodiment, the concentration of co-polyamino acid bearing carboxylate charges and hydrophobic radicals is at most 10 mg/mL.

In an embodiment, the concentration of co-polyamino acid bearing carboxylate charges and hydrophobic radicals is at most 5 mg/mL.

In an embodiment, the concentration of co-polyamino acid bearing carboxylate charges and hydrophobic radicals is at most 2.5 mg/mL.

In an embodiment, the compositions according to the invention comprise, in addition, a prandial insulin. The prandial insulins are soluble at pH 7.

A prandial insulin is understood to mean a so-called rapid or “regular” insulin.

The so-called rapid prandial insulins are insulins that must respond to the needs induced by the ingestion of proteins and saccharides during a meal; they must act in less than 30 minutes.

In an embodiment, the so-called “regular” prandial insulin is human insulin.

In an embodiment, the prandial insulin is a recombinant human insulin as described in the European Pharmacopoeia and the American Pharmacopoeia.

The human insulin is marketed under the trade names of Humulin® (ELI LILLY) and Novolin® (NOVO NORDISK), for example.

The so-called very rapid (fast acting) prandial insulins are insulins which are obtained by recombination and the primary structure of which was modified in order to decrease their time of action.

In an embodiment, the so-called very rapid (fast acting) prandial insulins are chosen in the group comprising the insulin lispro (Humalog®), insulin glulisine (Apidra®), and the insulin aspart (NovoLog®).

In an embodiment, the prandial insulin is insulin lispro.

In an embodiment, the prandial insulin is insulin glulisine.

In an embodiment, the prandial insulin is insulin aspart.

In an embodiment, the compositions according to the invention comprise in total from 60 to 800 U/mL of insulin with a combination of prandial insulin and of basal insulin of which the isoelectric point is from 5.8 to 8.5.

In an embodiment, the compositions according to the invention comprise in total from 100 to 500 U/mL of insulin with a combination of prandial insulin and of basal insulin of which the isoelectric point is from 5.8 to 8.5.

In an embodiment, the compositions according to the invention comprise in total 800 U/mL of insulin with a combination of prandial insulin and of basal insulin of which the isoelectric point is from 5.8 to 8.5.

In an embodiment, the compositions according to the invention comprise in total 700 U/mL of insulin with a combination of prandial insulin and of basal insulin of which the isoelectric point is from 5.8 to 8.5.

In an embodiment, the compositions according to the invention comprise in total 600 U/mL of insulin with a combination of prandial insulin and of basal insulin of which the isoelectric point is from 5.8 to 8.5.

In an embodiment, the compositions according to the invention comprise in total 500 U/mL of insulin with a combination of prandial insulin and of basal insulin of which the isoelectric point is from 5.8 to 8.5.

In an embodiment, the compositions according to the invention comprise in total 400 U/mL of insulin with a combination of prandial insulin and of basal insulin of which the isoelectric point is from 5.8 to 8.5.

In an embodiment, the compositions according to the invention comprise in total 300 U/mL of insulin with a combination of prandial insulin and of basal insulin of which the isoelectric point is from 5.8 to 8.5.

In an embodiment, the compositions according to the invention comprise in total 266 U/mL of insulin with a combination of prandial insulin and of basal insulin of which the isoelectric point is from 5.8 to 8.5.

In an embodiment, the compositions according to the invention comprise in total 200 U/mL of insulin with a combination of prandial insulin and of basal insulin of which the isoelectric point is from 5.8 to 8.5.

In an embodiment, the compositions according to the invention comprise in total 100 U/mL of insulin with a combination of prandial insulin and of basal insulin of which the isoelectric point is from 5.8 to 8.5.

The proportions between the basal insulin of which the isoelectric point is from 5.8 to 8.5, and the prandial insulin are, for example, in percentages, 25/75, 30/70, 40/60, 50/50, 60/40, 63/37 70/30, 75/25, 80/20, 83/17, 90/10 for formulations as described above comprising 60 to 800 U/mL. However, any other proportion can be used.

In an embodiment, the compositions according to the invention comprise, in addition, a gastrointestinal hormone.

“Gastrointestinal hormones” is understood to mean the hormones chosen from the group consisting of GLP-1 RA (glucagon like peptide-1 receptor agonist) and GIP (glucose-dependent insulinotropic peptide), oxyntomoduline (a proglucagon derivative), the peptide YY, amylin, cholecystokinin, the pancreatic polypeptide (PP), ghrelin and enterostatin, the analogs or derivatives thereof and/or the pharmaceutically acceptable salts thereof.

In an embodiment, the gastrointestinal hormones arc GLP-1 RA analogs or derivatives chosen from the group consisting of exenatide or Byetta® (ASTRA-ZENECA), liraglutide or Victoza® (NOVO NORDISK), lixisenatide or Lyxumia® (SANOFI), albiglutide or Tanzeurn® (GSK) or dulaglutide or Trulicity® (ELI LILLY & CO), the analogs or derivatives thereof and the pharmaceutically acceptable salts thereof.

In an embodiment, the gastrointestinal hormone is pramlintide or Symlin® (ASTRA-ZENECA).

In an embodiment, the gastrointestinal hormone is exenatide or Byetta®, the analogs or derivatives thereof and the pharmaceutically acceptable salts thereof.

In an embodiment, the gastrointestinal hormone is liraglutide or Victoza®, the analogs or derivatives thereof and the pharmaceutically acceptable salts thereof.

In an embodiment, the gastrointestinal hormone is lixisenatide or Lyxumia®, the analogs or derivatives thereof and the pharmaceutically acceptable salts thereof.

In an embodiment, the gastrointestinal hormone is albiglutide or Tanzeum®, the analogs or derivatives thereof and the pharmaceutically acceptable salts thereof.

In an embodiment, the gastrointestinal hormone is dulaglutide or Trulicity®, the analogs or derivatives thereof and the pharmaceutically acceptable salts thereof.

In an embodiment, the gastrointestinal hormone is pramlintide or Symlin®, the analogs or derivatives thereof and the pharmaceutically acceptable salts thereof.

“Analog,” when used in reference to a peptide or a protein, is understood to mean a peptide or a protein in which one or more constitutive residues of amino acids have been substituted by other residues of amino acids and/or in which one or more constitutive residues of amino acids have been removed and/or in which one or more constitutive residues of amino acids have been added. The admissible percentage of homology for the present definition of an analog is 50%.

“Derivative,” when used in reference to a peptide or a protein, is understood to mean a peptide or a protein or an analog which has been chemically modified by a substituent which is not present in the peptide or the protein or the analog of reference, that is to say a peptide or a protein which has been made by creation of covalent bonds, in order to introduce substituents.

In an embodiment, the substituent is chosen from the group consisting of fatty chains.

In an embodiment, the concentration of gastrointestinal hormone is in an interval from 0.01 to 100 mg/mL.

In an embodiment, the concentration of exenatide, the analogs or derivatives thereof and the pharmaceutically acceptable salts thereof is within an interval from 0.04 to 0.5 mg/mL.

In an embodiment, the concentration of liraglutide, the analogs or derivatives thereof and the pharmaceutically acceptable salts thereof is within an interval from 1 to 10 mg/mL.

In an embodiment, the concentration of lixisenatide, the analogs or derivatives thereof and the pharmaceutically acceptable salts thereof is within an interval from 0.01 to 1 mg/mL.

In an embodiment, the concentration of albiglutide, the analogs or derivatives thereof and the pharmaceutically acceptable salts thereof is from 5 to 100 mg/mL.

In an embodiment, the concentration of dulaglutide, the analogs or derivatives thereof and the pharmaceutically acceptable salts thereof is from 0.1 to 10 mg/mL.

In an embodiment, the concentration of pramlintide, the analogs or derivatives thereof and the pharmaceutically acceptable salts thereof is from 0.1 to 5 mg/mL.

In an embodiment, the compositions according to the invention are produced by mixing commercial solutions of basal insulin of which the isoelectric point is from 5.8 to 8.5, and commercial solutions of GLP-1 RA, of GLP-1 RA analog or derivative in volume ratios within an interval from 10/90 to 90/10.

In an embodiment, the composition according to the invention comprises a daily dose of basal insulin and a daily dose of gastrointestinal hormone.

In an embodiment, the compositions according to the invention comprise from 40 U/mL to 500 U/mL of basal insulin of which the isoelectric point is from 5.8 to 8.5, and from 0.05 to 0.5 mg/mL of exenatide.

In an embodiment, the compositions according to the invention comprise from 40 U/mL to 500 U/mL of basal insulin of which the isoelectric point is from 5.8 to 8.5, and from 1 to 10 mg/mL of liraglutide.

In an embodiment, the compositions according to the invention comprise from 40 U/mL to 500 U/mL of basal insulin of which the isoelectric point is from 5.8 to 8.5, and from 0.01 to 1 mg/mL of lixisenatide.

In an embodiment, the compositions according to the invention comprise from 40 U/mL to 500 U/mL of basal insulin of which the isoelectric point is from 5.8 to 8.5, and from 5 to 100 mg/mL of albiglutide.

In an embodiment, the compositions according to the invention comprise from 40 U/mL to 500 U/mL of basal insulin of which the isoelectric point is from 5.8 to 8.5, and from 0.1 to 10 mg/mL/of dulaglutide.

In an embodiment, the compositions according to the invention comprise 500 U/mL of basal insulin of which the isoelectric point is from 5.8 to 8.5, and from 0.04 to 0.5 mg/mL of exenatide.

In an embodiment, the compositions according to the invention comprise 500 U/mL of basal insulin of which the isoelectric point is from 5.8 to 8.5, and from 1 to 10 mg/mL of liraglutide.

In an embodiment, the compositions according to the invention comprise 500 U/mL of basal insulin of which the isoelectric point is from 5.8 to 8.5, and from 0.01 to 1 mg/mL of lixisenatide.

In an embodiment, the compositions according to the invention comprise 500 U/mL of basal insulin of which the isoelectric point is from 5.8 to 8.5, and from 5 to 100 mg/mL of albiglutide.

In an embodiment, the compositions according to the invention comprise 500 U/mL of basal insulin of which the isoelectric point is from 5.8 to 8.5, and from 0.1 to 10 mg/mL of dulagluide.

In an embodiment, the compositions according to the invention comprise 400 U/mL of basal insulin of which the isoelectric point is from 5.8 to 8.5, and from 0.04 to 0.5 mg/mL of exenatide.

In an embodiment, the compositions according to the invention comprise 400 U/mL of basal insulin of which the isoelectric point is from 5.8 to 8.5, and from 1 to 10 mg/mL of liraglutide.

In an embodiment, the compositions according to the invention comprise 400 U/mL of basal insulin of which the isoelectric point is from 5.8 to 8.5, and from 0.01 to 1 mg/mL of lixisenatide.

In an embodiment, the compositions according to the invention comprise 400 U/mL of basal insulin of which the isoelectric point is from 5.8 to 8.5, and from 5 to 100 mg/mL of albiglutide.

In an embodiment, the compositions according to the invention comprise 400 U/mL of basal insulin of which the isoelectric point is from 5.8 to 8.5, and from 0.1 to 10 mg/mL of dulaglutide.

In an embodiment, the compositions according to the invention comprise 300 U/mL of basal insulin of which the isoelectric point is from 5.8 to 8.5, and from 0.04 to 0.5 mg/mL, of exenatide.

In an embodiment, the compositions according to the invention comprise 300 U/mL of basal insulin of which the isoelectric point is from 5.8 to 8.5, and from 1 to 10 mg/mL of liraglutide.

In an embodiment, the compositions according to the invention comprise 300 U/mL of basal insulin of which the isoelectric point is from 5.8 to 8.5, and from 0,01 to 1 mg/mL of lixisenatide.

In an embodiment, the compositions according to the invention comprise 300 U/mL of basal insulin of which the isoelectric point is from 5.8 to 8.5, and from 5 to 100 mg/mL of albiglutide.

In an embodiment, the compositions according to the invention comprise 300 U/mL of basal insulin of which the isoelectric point is from 5.8 to 8.5, and from 0,1 to 10 mg/mL of dulaglutide.

In an embodiment, the compositions according to the invention comprise 225 U/mL of basal insulin of which the isoelectric point is from 5.8 to 8.5, and from 0.04 to 0.5 mg/mL of exenatide.

In an embodiment, the compositions according to the invention comprise 225 U/mL of basal insulin of which the isoelectric point is from 5.8 to 8.5, and from 1 to 10 mg/mL of liraglutide.

In an embodiment, the compositions according to the invention comprise 225 U/mL of basal insulin of which the isoelectric point is from 5.8 to 8.5, and from 0.01 to 1 mg/mL of lixisenatide.

In an embodiment, the compositions according to the invention comprise 225 U/mL of basal insulin of which the isoelectric point is from 5.8 to 8.5, and from 5 to 100 mg/mL of albiglutide.

In an embodiment, the compositions according to the invention comprise 225 U/mL of basal insulin of which the isoelectric point is from 5.8 to 8.5, and from 0.1 to 10 mg/mL of dulaglutide.

In an embodiment, the compositions according to the invention comprise 200 U/mL of basal insulin of which the isoelectric point is from 5.8 to 8.5, and from 0.04 to 0.5 mg/mL of exenatide.

In an embodiment, the compositions according to the invention comprise 200 U/mL of basal insulin of which the isoelectric point is from 5.8 to 8.5, and from 1 to 10 mg/mL of liraglutide.

In an embodiment, the compositions according to the invention comprise 200 U/mL of basal insulin of which the isoelectric point is from 5.8 to 8.5, and from 0.01 to 1 mg/mL of lixisenatide.

In an embodiment, the compositions according to the invention comprise 200 U/mL of basal insulin of which the isoelectric point is from 5.8 to 8.5, and from 5 to 100 mg/mL of albiglutide.

In an embodiment, the compositions according to the invention comprise 200 U/mL of basal insulin of which the isoelectric point is from 5.8 to 8.5, and from 0.1 to 10 mg/mL of dulaglutide.

In an embodiment, the compositions according to the invention comprise 100 U/mL (or approximately 3.6 mg/mL) of basal insulin of which the isoelectric point is from 5.8 to 8.5, and from 0.04 to 0.5 mg/mL of exenatide.

In an embodiment, the compositions according to the invention comprise 100 U/mL (or approximately 3.6 mg/mL) of basal insulin of which the isoelectric point is from 5.8 to 8.5, and from 1 to 10 mg/mL of liraglutide.

In an embodiment, the compositions according to the invention comprise 100 U/mL (or approximately 3.6 mg/mL) of basal insulin of which the isoelectric point is from 5.8 to 8.5, and from 0.01 to 1 mg/mL of lixisenatide.

In an embodiment, the compositions according to the invention comprise 100 U/mL of basal insulin of which the isoelectric point is from 5.8 to 8.5, and from 5 to 100 mg/mL of albiglutide.

In an embodiment, the compositions according to the invention comprise 100 U/mL of basal insulin of which the isoelectric point is from 5.8 to 8.5, and from 0.1 to 10 mg/mL of dulaglutide.

In an embodiment, the compositions according to the invention comprise 40 U/mL of basal insulin of which the isoelectric point is from 5.8 to 8.5, and from 0.04 to 0.5 mg/mL of exenatide.

In an embodiment, the compositions according to the invention comprise 40 U/mL of basal insulin of which the isoelectric point is from 5.8 to 8.5, and from 1 to 10 mg/mL, of liraglutide.

In an embodiment, the compositions according to the invention comprise 40 U/mL of basal insulin of which the isoelectric point is from 5.8 to 8.5, and from 0.01 to 1 mg/mL of lixisenatide.

In an embodiment, the compositions according to the invention comprise 40 U/mL of basal insulin of which the isoelectric point is from 5.8 to 8.5, and from 5 to 100 mg/mL of albiglutide.

In an embodiment, the compositions according to the invention comprise 40 U/mL of basal insulin of which the isoelectric point is from 5.8 to 8.5, and from 0.1 to 10 mg/mL of dulaglutide.

In an embodiment, the compositions according to the invention furthermore comprise zinc salts at a concentration from 0 to 5000 μM.

In an embodiment, the compositions according to the invention furthermore comprise zinc salts at a concentration from 0 to 4000 μM.

In an embodiment, the compositions according to the invention furthermore comprise zinc salts at a concentration from 0 to 3000 μM.

In an embodiment, the compositions according to the invention furthermore comprise zinc salts at a concentration from 0 to 2000 μM.

In an embodiment, the compositions according to the invention furthermore comprise zinc salts at a concentration from 0 to 1000 μM.

In an embodiment, the compositions according to the invention furthermore comprise zinc salts at a concentration from 50 to 600 μM.

In an embodiment, the compositions according to the invention furthermore comprise zinc salts at a concentration from 100 to 500 μM.

In an embodiment, the compositions according to the invention furthermore comprise zinc salts at a concentration from 200 to 500 μM.

In an embodiment, the compositions according to the invention furthermore comprise buffers.

In an embodiment, the compositions according to the invention comprise buffers at concentrations from 0 to 100 mM.

In an embodiment, the compositions according to the invention comprise buffers at concentrations from 15 to 50 mM.

In an embodiment, the compositions according to the invention comprise a buffer chosen from the group consisting of a phosphate buffer, tris (trishydroxymethylaminomethane) and sodium citrate.

In an embodiment, the buffer is sodium phosphate.

In an embodiment, the buffer is tris (trishydroxymethylaminomethane).

In an embodiment, the buffer is sodium citrate.

In an embodiment, the compositions according to the invention furthermore comprise preservatives.

In an embodiment, the preservatives are chosen from the group consisting of m-cresol and phenol, alone or in a mixture.

In an embodiment, the concentration of preservatives is from 10 to 50 mM.

In an embodiment, the concentration of preservatives is from 10 to 40 mM.

In an embodiment, the compositions according to the invention furthermore comprise a surfactant.

In an embodiment, the surfactant is chosen from the group consisting of propylene glycol and polysorbate.

The compositions according to the invention can furthermore comprise additives such as tonicity agents.

In an embodiment, the tonicity agents are chosen from the group consisting of glycerol, sodium chloride, mannitol and glycine.

The compositions according to the invention can comprise, in addition, all the excipients in conformity with the pharmacopoeias and compatible with the insulins used at the conventional concentrations.

The invention also relates to a pharmaceutical formulation according to the invention, characterized in that it is obtained by drying and/or lyophilization.

In the case of local and systemic releases, the modes of administration considered are by intravenous, subcutaneous, intradermal or intramuscular route.

The transdermal, oral, nasal, vaginal, ocular, buccal, pulmonary routes of administration are also considered.

In an embodiment, the composition according to the invention is characterized in that it is administered 1 time a day.

In an embodiment, the composition according to the invention is characterized in that it is administered at least 2 times a day.

In an embodiment, the composition according to the invention is characterized in that it is administered 2 times a day.

In an embodiment, the composition according to the invention is characterized in that it further includes a prandial insulin.

In an embodiment, the composition according to the invention further including at least one prandial insulin is characterized in that it is administered 1 time a day.

In an embodiment, the composition according to the invention further including at least one prandial insulin is characterized in that it is administered at least 2 times a day.

In an embodiment, the composition according to the invention further including at least one prandial insulin is characterized in that it is administered 2 times a day.

In an embodiment, the composition according to the invention is characterized in that it further includes a gastrointestinal hormone.

In an embodiment, the composition according to the invention moreover including at least one gastrointestinal hormone is characterized in that it is administered 1 time a day.

In an embodiment, the composition according to the invention further including at least one gastrointestinal hormone is characterized in that it is administered at least 2 times a day.

In an embodiment, the composition according to the invention further including at least one gastrointestinal hormone is characterized in that it is administered 2 times a day.

In an embodiment, the composition according to the invention is characterized in that the gastrointestinal hormone is a GLP-1 RA.

In an embodiment, the composition according to the invention further including at least one GLP-1 RA is characterized in that it is administered 1 time a day.

In an embodiment, the composition according to the invention further including at least one GLP-1 RA is characterized in that it is administered at least 2 times a day.

In an embodiment, the composition according to the invention further including at least one GLP-1 RA is characterized in that it is administered 2 times a day.

The invention also relates to single-dose formulations at a pH from 6,0 to 8.0, comprising a basal insulin the isoelectric point of which is from 5.8 to 8.5.

The invention also relates to single-dose formulations at a pH from 6.0 to 8.0, comprising a basal insulin of which the isoelectric point is from 5.8 to 8.5, and a prandial insulin.

The invention also relates to single-dose formulations at a pH from 6.0 to 8.0, comprising a basal insulin of which the isoelectric point is from 5.8 to 8.5, and a gastrointestinal hormone as defined above.

The invention also relates to single-dose formulations at a pH from 6.0 to 8.0, comprising a basal insulin of which the isoelectric point is from 5.8 to 8.5, a prandial insulin, and a gastrointestinal hormone as defined above.

The invention also relates to single-dose formulations at a pH from 6.6 to 7.8, comprising a basal insulin of which the isoelectric point is from 5.8 to 8.5.

The invention also relates to single-dose formulations at a pH from 6.6 to 7.8, comprising a basal insulin the isoelectric point of which is from 5.8 to 8.5, and a prandial insulin.

The invention also relates to single-dose formulations at a pH from 6.6 to 7.8, comprising a basal insulin of which the isoelectric point is from 5.8 to 8.5, and a gastrointestinal hormone as defined above.

The invention also relates to single-dose formulations at a pH from 6.6 to 7.8, comprising a basal insulin of which the isoelectric point is from 5.8 to 8.5, a prandial insulin, and a gastrointestinal hormone as defined above.

The invention also relates to single-dose formulations at a pH from 6.6 to 7.6, comprising a basal insulin of which the isoelectric point is from 5.8 to 8.5.

The invention also relates to single-dose formulations at a pH from 6.6 to 7.6, comprising a basal insulin the isoelectric point of which is from 5.8 to 8.5, and a prandial insulin.

The invention also relates to single-dose formulations at a pH from 6.6 to 7.6, comprising a basal insulin of which the isoelectric point is from 5.8 to 8.5, and a gastrointestinal hormone as defined above.

The invention also relates to single-dose formulations at a pH from 6.6 to 7.6, comprising a basal insulin of which the isoelectric point is from 5.8 to 8.5, a prandial insulin, and a gastrointestinal hormone as defined above.

In an embodiment, the single-dose formulations comprise, in addition, a co-polyamino acid as defined above.

In an embodiment, the formulations are in the form of an injectable solution.

In an embodiment, the basal insulin of which the isoelectric point is from 5.8 to 8.5, is insulin glargine.

In an embodiment, the prandial insulin is human insulin.

In an embodiment, the insulin is a recombinant human insulin as described in the European Pharmacopoeia and the American Pharmacopoeia.

In an embodiment, the prandial insulin is chosen from the group comprising insulin lispro (Humalog®), insulin glulisine (Apidra®) and insulin aspart (NovoLog®).

In an embodiment, the prandial insulin is insulin lispro.

In an embodiment, the prandial insulin is insulin glulisine.

In an embodiment, the prandial insulin is insulin aspart,

In an embodiment, the GLP-1 RA, the GLP-1 RA analog or derivative is chosen from the group comprising exenatide (Byetta®), liraglutide (Victoza®), lixisenatide (Lyxumia®), albiglutide (Tanzeum®), dulaglutide (Trulicity®), or one of the derivatives thereof.

In an embodiment, the gastrointestinal hormone is exenatide.

In an embodiment, the gastrointestinal hormone is liraglutide.

In an embodiment, the gastrointestinal hormone is lixisenatide.

In an embodiment, the gastrointestinal hormone is albiglutide.

In an embodiment, the gastrointestinal hormone is dulaglutide.

The solubilization at a pi-I from 6.0 to 8.0 of the basal insulins, the isoelectric point of which is from 5.8 to 8.5, by the co-polyamino acids bearing carboxylate charges and at least one hydrophobic radical according to the invention can be observed and controlled simply with the naked eye by a change in the appearance of the solution.

The solubilization at a pH from 6.6 to 7.8 of the basal insulins, the isoelectric point of which is from 5.8 to 8.5, by the co-polyamino acids bearing carboxylate charges and at least one hydrophobic radical according to the invention can be observed and controlled simply with the naked eye by a change in the appearance of the solution.

Moreover and as importantly, the applicant was able to verify that a basal insulin of which the isoelectric point is from 5.8 to 8.5, solubilized at a pH from 6.0 to 8.0 in the presence of a co-polyamino acid bearing carboxylate charges and at least one hydrophobic radical according to the invention maintains its slow-acting insulin action whether alone or in combination with a prandial insulin or a gastrointestinal hormone.

The applicant was also able to verify that a prandial insulin mixed at a pH from 6.0 to 8.0 in the presence of a co-polyamino acid bearing carboxylate charges and at least one hydrophobic radical according to the invention and of a basal insulin of which the isoelectric point is from 5.8 to 8.5 maintains its rapid-acting insulin action.

The preparation of a composition according to the invention has the advantage that it can be carried out by simply mixing an aqueous solution of basal insulin of which the isoelectric point is from 5.8 to 8.5, and a co-polyamino acid bearing carboxylate charges and at least one hydrophobic radical according to the invention, in aqueous solution or in lyophilized form. If necessary, the pH of the preparation is adjusted to a pH from 6 to 8.

The preparation of a composition according to the invention has the advantage that it can be carried out by simply mixing an aqueous solution of basal insulin of which the isoelectric point is from 5.8 to 8.5, a solution of prandial insulin, and a co-polyamino acid bearing carboxylate charges and at least one hydrophobic radical according to the invention, in aqueous solution or in lyophilized form. If necessary, the pH of the preparation is adjusted to a pH from 6 to 8.

The preparation of a composition according to the invention has the advantage that it can be carried out by simply mixing an aqueous solution of basal insulin of which the isoelectric point is from 5.8 to 8.5, a solution of GLP-1 RA, a GLP-1 RA analog or derivative, and a co-polyamino acid bearing carboxylate charges and at least one hydrophobic radical according to the invention, in aqueous solution or in lyophilized form. If necessary, the pH of the preparation is adjusted to a pH from 6 to 8.

The preparation of a composition according to the invention has the advantage that it can be carried out by simply mixing an aqueous solution of basal insulin of which the isoelectric point is from 5.8 to 8.5, a solution of prandial insulin, a solution of GLP-1 RA, a GLP-1 RA analog or derivative, and a co-polyamino acid bearing carboxylate charges and at least one hydrophobic radical according to the invention, in aqueous solution or in lyophilized form. If necessary, the pH of the preparation is adjusted to a pH from 6 to 8.

In an embodiment, the mixture of basal insulin and co-polyamide is concentrated by ultrafiltration before the mixing with prandial insulin in aqueous solution or in lyophilized form.

If necessary, the composition of the mixture is adjusted with excipients such as glycerol, zinc chloride and polysorbate (Tween®) by adding concentrated solutions of these excipients within the mixture. If necessary, the pH of the preparation is adjusted to a pH from 6 to 8.

### EXAMPLES

The invention is described in greater detail in reference to the following examples in a non-limiting manner.

### Part A

AA: Synthesis of the Hydrophobic Molecules in which p=1

The hydrophobic radicals are represented in the following table by the corresponding hydrophobic molecule before grafting onto the co-polyamino acid.

Table 1LA
list and structures of the hydrophobic molecules synthesized according to the
invention,.

| | Structure of the hydrophobic molecule before grafting onto the co- |
|---|---|
| No. | polyamino acid |
| AA1 | 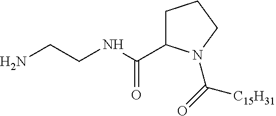 |
| AA2 | 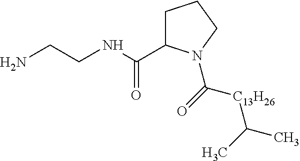 |
| AA3 | 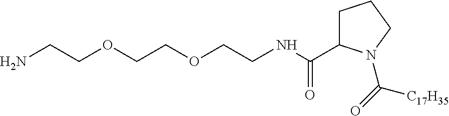 |
| AA4 | 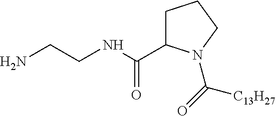 |
| AA5 | 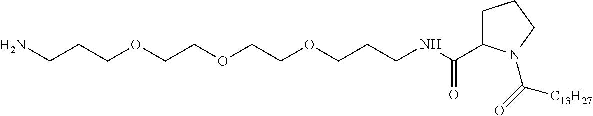 |
| AA6 | 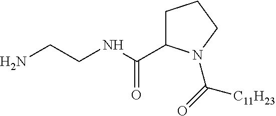 |
| AA7 | 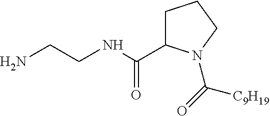 |
| AA8 | 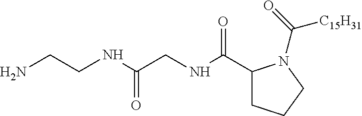 |
| AA9 | 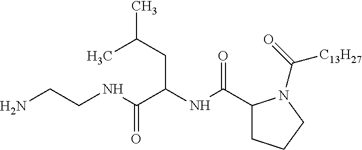 |

### Example AA1

### Molecule AA1

Molecule A1: Product obtained by the reaction between palmitoyl chloride and L-proline.

A solution of palmitoyl chloride (23.0 g, 83.7 mmol) in acetone (167 mL) is added dropwise over 90 min to a solution of L-proline (10.6 g, 92.1 mmol) in 1 N aqueous sodium hydroxide (230 mL; 230 mmol). After 14 h of stirring at room temperature, the heterogeneous mixture is cooled to 0° C., then filtered on a sintered frit to give a white solid which is washed with water (2×100 mL), then with diisopropyl ether (100 mL). The solid is dried at reduced pressure. The solid is then dissolved at reflux in 200 mL of water, then 8 mL of a 37% hydrochloric acid solution are added until obtaining pH=1. The opalescent reaction medium is then cooled to 0° C. The precipitate obtained is filtered through a sintered filter, then washed with water (5×50 mL) until filtrates having a pH from 6.0 to 8.0 are obtained, then it is dried overnight at 50° C. in an oven under vacuum. The product is purified by recrystallization in diisopropyl ether. A white solid is obtained.
Yield: 22.7 g (77%).

¹H NMR (CDCl₃, ppm): 0.88 (3H); 1.19-1.45 (24H); 1.58-1.74 (2H); 1.88-2.14 (3H); 2.15-2.54 (3H), 3.47 (1H); 3.58 (1H); 4.41 (0.1H); 4.61 (0.9H) 6.60-8.60 (1H).

Molecule A2: Product obtained by reaction between molecule A1 and N-Boc-1-ethylenediamine.

N,N-diisopropylethylamine (DIPEA) (68.8 g, 532.3 mmol), 1-hydroxybenzotriazole (IIOBt) (37.1 g, 274.6 mmol), then N-(3-dimethylaminopropyl)-N′-ethylcarbodiimide (EDC) (53.1 g, 277.0 mmol) are added successively at room temperature to a solution of molecule A1 (75.1 g, 212.4 mmol) in 1500 mL of chloroform. After 15 min of stirring at room temperature, a solution of N-Boc-ethylenediamine (BocEDA) (37.6 g, 234.7 mmol) in 35 mL of chloroform is added. After 18 h of stirring at room temperature, a 0.1 N HCl solution (2.1 L), then a saturated NaCl solution (1 L) are added. The phases are separated, then the organic phase is washed successively with a 0.1 N HCl/saturated NaCl (2.1 L/1 L), a saturated NaCl solution (2 L), a saturated NaHCO3 solution (2 L), then a saturated NaCl solution (2 L). The organic phase is dried over anhydrous sodium sulfate, filtered then concentrated at reduced pressure. The solid obtained is purified by triturations in dlisopropyl ether (3×400 mL), to give a solid after drying under vacuum at 40° C.
Yield: 90.4 g (86%).

¹H NMR (CDCl₃, ppm): 0.88 (3H); 1.20-1.37 (24H); 1.44 (9H); 1.54-1.70 (2H); 1.79-1.92 (1H); 1.92-2.04 (1H); 2.03-2.17 (1H); 2.17-2.44 (3H); 3.14-3.36 (4H); 3.43 (1H); 3.56 (1H); 4.29 (0.1H); 4.51 (0.9H); 4.82 (0.1H); 5.02 (0.9H); 6.84 (0.1H); 7.22 (0.9H).

Molecule AA1

At 0° C. a 4 N hydrochloric acid solution in dioxane (100 mL, 400 mmol) is added dropwise to a solution of molecule A2 (20.1 g, 40.5 mmol) in 330 mL of dichloromethane. After 3 h 30 of stirring at room temperature, the solution is concentrated at reduced pressure. The residue is purified by flash chromatography (methanol, dichloromethane) to give a white solid of molecule AA1 in hydrochloride salt form.
Yield: 16.3 g (93%)

¹H NMR (CDCl₃, ppm): 0.88 (3H); 1.07-1.40 (24H); 1.49-1.63 (2H); 1.77-2.18 (4H); 2.18-2.45 (2H); 3.14-3.32 (2H); 3.42-3.63 (2H); 3.63-3.84 (2H); 4.37 (0.1H); 4.48 (0.9H); 6.81-8.81 (4H).

LC/MS (BSI): 396.5; (calculated ([M+H]⁺): 396.4).

### Example AA2

### Molecule AA2

Molecule A3: 15-methylhexadecan-1-ol,

Magnesium chips (9.46 g, 389 mmol) are introduced into a three-neck round-bottom flask under argon. The magnesium is covered with anhydrous THF (40 mL) and a few drops of 1-promo-3-methylbutane are added at room temperature to initiate the reaction. After the observation of an exotherm and slight turbidity of the medium, the rest of the 1-bromo-3-methylbutane (53.87 g, 357 mmol) is added dropwise over 90 min while the temperature of the medium remains stable from 50 to 60° C. The reaction mixture is then heated at 70° C. for 2 h.

In a three-neck round-bottom flask under argon, a solution of 12-bromo-1-dodecanol (43 g, 162.1 mmol) in THF (60 mL) is added dropwise to a solution of CuCl (482 mg, 4.86 mmol) dissolved in NMP (62 mL) at 0° C. To this solution, the freshly prepared hot organomagnesium solution is then added dropwise in such a manner as to maintain the temperature of the medium below 20° C. The mixture is then stirred at room temperature for 16 h. The medium is cooled to 0° C., and the reaction is stopped by addition of a 1 N aqueous HCl solution until the pH is 1, and the medium is extracted with ethyl acetate. After washing the organic phase with a saturated NaCl solution and drying over Na2SO4, the solution is filtered and concentrated under vacuum to yield an oil. After purification by DCVC on silica gel (cyclohexane, ethyl acetate), an oil which crystallizes at room temperature is obtained.
Yield: 32.8 g (74%)

¹NMR (CDCl₃, ppm): 0.87 (6H); 1.14 (2H); 1.20-1.35 (22H); 1.50-1.55 (3H); 3.64 (2H).

Molecule A4: 15-methylnexadecanole Acid

Potassium permanganate (38.2 g, 241.5 mmol) is added in small portions to a solution of molecule A3 (20.65 g, 80.5 mmol) and tetrabutylammonium bromide (14.02 g, 42.5 mmol) in a mixture of acetic acid/dichloroethane/water (124/400/320 mL) at a room temperature. After stirring at reflux for 5 h and return to room temperature, the medium is acidified to pH I by gradual addition of 5 N HCl. Na2SO3 (44.6 g, 354.3 mmol) is then gradually added until the medium is bleached. The aqueous phase is extracted with dichloromethane and the combined organic phases are dried over Na2SO4, filtered and concentrated under vacuum. After purification by chromatography on silica gel (cyclohexane, ethyl acetate, acetic acid), a white solid is obtained.
Yield: 19.1 g (quantitative)

¹H NMR (CDCl₃, ppm): 0.87 (6H); 1.14 (2H); 1.22-1.38 (20H); 1.51 (1H); 1.63 (2H); 2.35 (2H).

### Molecule A5: Product Obtained by the Reaction Between Molecule A4 and L-Proline.

Dicyclohexyl carbodiimide (DCC) (8.01 g, 38.8 mmol) and N-hydroxysuccinimide (NHS) (4.47 g, 38.8 mmol) are added successively to a solution of molecule A4 (10 g, 37 mmol) in THF (360 mL) at 0° C. After 17 h of stirring at room temperature, the medium is cooled to 0° C. for 20 min, filtered on a sintered filter, L-proline (4 g, 37.7 mmol), triethylamine (TEA, 34 mL) and water (30 mL) are added to the filtrate. After 20 h of stirring at room temperature, the medium is treated with a 1 N aqueous HCl solution until the pH is 1. The aqueous phase is extracted with dichloromethane (2×125 mL). The combined organic phases are washed with a 1 N aqueous HCl solution (2×100 (mL), water (100 mL), then a saturated aqueous NaCl solution (100 mL). After drying over Na2SO4, the organic phase is filtered, concentrated under a vacuum, and the residue is purified by chromatography on silica gel (cyclohexane, ethyl acetate, acetic acid)
Yield: 9.2 g (72%)

¹H NMR (CDCl₃, ppm): 0.86 (6H); 1.14 (2H); 1.22-1.38 (20H); 1.50 (1H); 1.67 (2H); 1.95-2.10 (3H); 2.34 (2H); 2.49 (1H); 3.47 (1H); 3.56 (1H); 4.61 (1H).
LC/MS (ESI): 368.3; (calculated (M+H]⁺): 368.6).

### Molecule A6: Product Obtained by the Reaction Between Molecule A5 and N-Boc-ethylenediamine.

TEA (5.23 mL) and 2-(1H-benzotriazol-1-yl)-1,1,3,3-tetramethyluronium tetrafluoroborate (TBTU) are added at room temperature to a solution of molecule A5 (9.22 g, 25.08 mmol) in a mixture of THF/DMF (200/50 mL). After 10 min of stirring, BocEDA (4.42 g, 27.6 mmol) is added. After stirring at room temperature for 17 h, the mixture is diluted with water (300 mL) at 0° C. and stirred for 20 min. The precipitate formed is filtered on a sintered filter, the filtrate is extracted with ethyl acetate. The combined organic phases are washed with a saturated NaHCO3 solution, dried over Na2SO4, filtered, concentrated under vacuum, and the residue is purified by flash chromatography (ethyl acetate, methanol).
Yield: 6.9 g (54%)

¹H NMR (CDCl₃, ppm): 0.86 (6H); 1.15 (2H); 1.22-1.38 (20H); 1.43 (9H); 1.50 (1H); 1.64 (4H); 1.85 (1H); 1.95 (1H); 2.10 (1H); 2.31 (2H); 3.20-3.35 (3H); 3.45 (1H); 3.56 (1H); 4.51 (1H); 5.05 (1H); 7.24 (1H).
LC/MS (ESI): 510.6; (calculated (M+H]⁺): 510.8).

### Molecule AA2

A 4 N HCl solution in dioxane (13 mL) is added to a solution of molecule A6 (5.3 g, 10.40 mmol) in dichloromethane (50 mL) at 0° C. After 5 h of stirring at 0° C., the medium is concentrated under vacuum, solubilized in water and lyophilized to give a white solid of molecule AA2 in hydrochloride salt form.
Yield: 4.6 g (99%)

¹H NMR (D₂O, ppm): 0.91 (6H); 1.22 (2H); 1.22-1.50 (20H); 1.63 (3H); 1.98 (1H); 2.10 (2H); 2.26 (1H); 2.39 (1H); 2.43 (1H); 3.22 (2H); 3.45-3.60 (3H); 3.78 (1H); 4.42 (1H).
LC/MS (ESI): 410.4; (calculated ([M+H]⁺): 410.7).

### Example AA3

### Molecule AA3

### Molecule A7: Product Obtained by the Reaction Between Stearoyl Chloride and L-Proline.

By a method similar to the one used for the preparation of molecule A1 applied to L-proline (5.0 g, 43.4 mmol) and to stearoyl chloride (12.0 g, 39.6 mmol), a white solid is obtained after purification by flash chromatography (methanol, dichloromethane).
Yield: 5.37 g (36%)

¹H NMR (CDCl₃, ppm): 0.88 (3H); 1.26-1.37 (28H); 1.64-1.70 (2H); 1.88-2.10 (3H); 2.36 (2H); 2.54-2.58 (1H); 3.46 (1H); 3.56 (1H); 4.62 (1H).
LC/MS (ESI): 382.6; (calculated ([M+H])⁺): 382.3).

### Molecule A8: Product Obtained by the Reaction Between Molecule A7 and N-Boc-tri(ethyleneglycol)diamine.

By a method similar to the one used for the preparation of molecule A6 applied to molecule A8 (33.81 g, 88.6 mmol) and to N-Boc-tri(ethyleneglycol)diamine (26.4 g, 106.3 mmol) in THF using DIPEA instead of TEA, a white solid is obtained after purification by flash chromatography (ethyl acetate, methanol).
Yield: 43.3 g (80%)

¹H NMR (CDCl₃, ppm): 0.87 (3H); 1.24 (30H); 1.43 (9H); 1.61 (2H); 1.82 (1H); 1.96 (1H); 2.25-2.45 (2H); 3.25-3.65 (14H); 4.30 (0.15H); 4.53 (0.85H); 5.25 (1H); 6.43 (0.15H); 7.25 (0.85H).
LC/MS (ESI): 612.6; (calculated ([M+H]⁺): 612.9).

### Molecule AA3

By a method similar to the one used for the preparation of molecule AA2 applied to molecule A8 (43 g, 70.3 mmol), the residue obtained after concentration under a vacuum is triturated in acetonitrile. The suspension is filtered and the solid is washed with acetonitrile and then with acetone. After drying under a vacuum, a white solid of molecule AA3 in the form of a hydrochloride salt is obtained.
Yield: 31.2 g (81%)

¹H NMR (DMSO-d₆, ppm): 0.85 (3H); 1.23 (28H); 1.45 (2H); 1.70-2.05 (4H); 2.13 (1H); 2.24 (1H); 2.95 (2H); 3.10-3.25 (2H); 3.30-3.65 (10H); 4.20-4.45 (1H); 7.85-8.25 (4H).
LC/MS (ESI): 512.4; (calculated ([M+H]⁺): 512.8).

### Example AA4

### Molecule AA4

### Molecule A9: Product Obtained by the Reaction Between Myristoyl Chloride and L-Proline

Myristoyl chloride (322 g, 1.30 mol) in solution in dichloromethane (DCM, 1.63 L) is added slowly over 1 hour to a solution of L-proline (300.40 g, 2.61 mol) in 2 N aqueous sodium hydroxide (1.63 L) at 0° C. At the end of the addition, the reaction medium is heated again to 20° C. over 3 h, then stirred for a further 2 h. The mixture is cooled to 0° C., and then a 37% HCl aqueous solution (215 mL) is added over 15 minutes. The reaction mixture is stirred for 1 h from 0° C. to 20° C. The organic phase is separated, washed with a 10% aqueous HCl solution (3×430 mL), a saturated aqueous NaCl solution (430 mL), dried over Na2SO4, filtered through cotton, then concentrated at reduced pressure. The residue is solubilized in heptane (1.31 L) at 50° C., then the solution is gradually brought to room temperature. After initiation of the crystallization using a glass rod, the medium is heated again at 40° C. for 30 min and then returned to room temperature over 4 h. A white solid is obtained after filtration on a sintered filter, washing with heptane (2×350 mL) and drying under reduced pressure.

Yield: 410 g (97%)

¹H NMR (CDCl₃, ppm): 0.88 (3H); 1.28 (20H); 1.70 (2H); 1.90-2.10 (3H); 2.36 (2H); 2.51 (1H); 3.47 (1H); 3.56 (1H); 4.61 (1H).

LC/MS (ESI): 326.4; 651.7; (calculated ([M+H]⁺): 326.3; ([2M+H]⁺): 651.6).

### Molecule A10: Product Obtained by the Reaction Between Molecule A9 and N-Boc-ethylenediamine

HOBt (8.94 g, 58.37 mmol), then BocEDA (112.20 g, 700.00 mmol) in solution in DCM (150 mL) are added successively to a solution of molecule A9 (190.00 g, 583.73 mmol) at 0° C. in DCM (2.9 L). EDC (123.10 g, 642.00 mmol) is added, and then the mixture is stirred for 17 h at front 0° C. to room temperature. The reaction mixture is then washed with a saturated aqueous NaHCO3 solution (2×1.5 L), a 1 N aqueous HCl solution (2×1.5 L) and then saturated aqueous NaCl solution (1.5 L), dried over Na2SO4, filtered and concentrated at reduced pressure. A white solid is obtained after recrystallization in acetonitrile.
Yield: 256.50 g (93%)

¹H NMR (CDCl₃, ppm): 0.88 (3H); 1.16-1.38 (20H); 1.44 (9H); 1.56-1.71 (2H); 1:78-2.45 (6H); 3.11-3.72 (6H); 4.30 (0.1H); 4.51 (0.9H); 4.87 (0.1H); 5.04 (0.9H); 6.87 (0.1H); 7.23 (0.9H).
LC/MS (EST): 468.0; (calculated ([M+H]⁺): 468.4).

### Molecule AA4

According to a method similar to the one used for the preparation of molecule AA1 applied to molecule A10 (256.50 g, 548.43 mmol), a white solid of molecule AA4 in hydrochloride salt form is obtained by trituration in pentane (1.6 L) and drying under reduced pressure at 40° C.

Yield: 220.00 g (99%)

¹H NMR (MeOD-d4, ppm): 0.90 (3H); 1.21-1.43 (20H); 1.54-1.66 (2H); 1.85-2.28 (4H); 2.39 (2H); 3.00-3.17 (2H); 3.30-3.40 (1H); 3.43-3.71 (3H); 4.29 (0.94H); 4.48 (0.06H).

LC/MS (ESI): 368.2; (calculated ([M+H]⁺): 368.3).

### Example AA5

### Molecule AA5

Molecule: A11: Product Obtained by the Reaction Between Molecule A9 and Boc-1 -amino-4,7,10-trioxa-13 -tridecaneamine.

By a method similar to the one used for the preparation of molecule A10 applied to molecule A9 (24.00 g, 73.73 mmol) and to Boc-1-amino-4,7,10-trioxa-l3-tridecaneamine (28.35 g, 88.48 mmol), an orange oil of molecule A11 is obtained.
Yield: 44.50 g (96%)

¹H NMR (CDCl₃, ppm): 0.87 (3H); 1.08-1.56 (20H); 1.43 (9H); 1.58-1.67 (2H); 1.70-2.00 (6H); 2.04-2.41 (4H); 3.16-3.77 (18H); 4.26-4.29 (0.2H); 4.50-4.54 (0.8H); 4.68-5.10 (1H); 6.74 (0.2H); 7.19 (0.8H)
LC/MS (ESI): 628.4; (calculated ([M+H]⁺): 628.5),

### Molecule AA5

According to a method similar to the one used for the preparation of molecule AA1 applied to molecule A11 (43.40 g, 69.12 mmol), a white solid of molecule AA5 in hydrochloride salt form is obtained after trituration 3 times in diethyl ether, solubilization of the residue in water, and lyophilization.

Yield: 38.70 g (98%)

¹H NMR (DMSO-d6, ppm): 0.85 (3H); 1.07-1.38 (20H); 1.41-1.52 (2H); 1.55-1.66 (2H); 1.70-2.02 (6H); 2.08-2.30 (2H); 2.78-2.87 (2H); 3.00-3.16 (2H); 3.29-3.66 (14H); 4.16-4.22 (0.65 H); 4.25-4.30 (0.35H); 7.74 (0.65H); 7.86 (3H); 8.10 (0.35H).

LC/MS (ESI): 528.4; (calculated ([M+H]⁺): 528.4).

### Example AA6

### Molecule AA6

### Molecule A12: Product Obtained by the Reaction Between Lauric Acid and L-Proline

DCC (12.83 g, 62.18 mmol) and N-hydroxysuccinimide (NHS) (7.16 g, 62.18 mmol) are added successively to a solution of lauric acid (11.86 g, 59.22 mmol) in THF (600 mL) at 0° C. After 18 h of stirring at room temperature, the medium is cooled to 0° C. for 20 min, filtered on a sintered filter. L-proline (7.5 g, 65.14 mmol), triethylamine (58.26 mL) and water (70 mL) are added to the filtrate. After 60 h of stirring at room temperature, the medium is diluted with water (250 mL). The aqueous phase is washed with ethyl acetate (2×200 mL), acidified to pH ˜1 with a 1 N aqueous HCl solution, then extracted with dichloromethane (3×150 mL). The combined organic phases are dried on Na2SO4, filtered, and concentrated under reduced pressure. A yellow oil of molecule A12 is obtained.

Yield: 14.85 g (84%)

¹H NMR (CDCl₃ppm); 0.87 (3H); 1.26 (16H); 1.70 (2H); 1.90-2.10 (3H); 2.35 (2H); 2.49 (1H), 3.48 (1H); 3.56 (1H), 4.60 (1H).

LC/MS (ESI): 298.3; (calculated ([M+H]⁺): 298.4).

### Molecule A 13: Product Obtained by the Reaction Between Molecule A12 and N-Boc-ethylenediamine.

By a method similar to the one used for the preparation of molecule A10 applied to molecule A12 (12.00 g, 40.35 mmol) and to BocEDA (7.76 g, 48.42 mmol), a colorless oil is obtained and used without other purification.

Yield: 17.40 g (94%)

1H NMR (CDCl3; ppm): 0.86 (3H); 1.11-1.68 (18H); 1.41 (9H); 1.80-2.38 (6H); 3.06-3.35 (4H); 3.37-3.49 (1H), 3.51-3.73 (1H); 4.26-4.31 (0.1H); 4.45-4.52 (0.9H); 4.91-5.19 (1H); 6.97 (0.1H); 7.23 (0.9H).

LC/MS (ESI): 440.4; (calculated ([M+H]+): 440.3).

### Molecule AA6

According to a method similar to the one used for the preparation of molecule AA1 applied to molecule A13 (8.85 g, 20.13 mmol), a white solid of molecule AA6 is obtained after basic washing, concentration under reduced pressure, then recrystallization in acetonitrile.
Yield: 6.53 g (96%)

1H NMR (DMSO, ppm): 0.85 (3H); 1.07-1.56 (20H); 1.68-2.03 (4H); 2.09-2.29 (2H); 2.50-2.58 (2H); 2.96-3.11 (2H); 3.21-3.59 (2H); 4.17-4.21 (0.65H); 4.25-4.29 (0.35H); 7.68 (0.65H); 8.00 (0.35H).
LC/MS (ESI): 340.3; (calculated ([M+H]+): 340.3).

### Example AA7

### Molecule AA7

### Molecule A14: Product Obtained by the Reaction Between Decanoic Acid and L-Proline

A decanoyl chloride solution (75.0 g, 393.27 mmol) in DCM (490 mL) is added to a solution of L-proline (90.5 g, 786.53 mmol) in 2 M sodium hydroxide (492 mL) at 0° C. and under stirring over a period of 35 minutes. After 16 h of stirring at from 0° C. to room temperature, the reaction mixture is cooled to 0° C., then a 37% aqueous HCl solution (65 mL) is added over 15 min. The mixture is stirred for 10 min at cold temperature, then for 30 min while raising the temperature to room temperature. After separation of the phases, the organic phase is washed with a 10% aqueous HCl solution (3×125 mL), then a saturated aqueous NaCl solution (125 mL), dried over Na2SO4, filtered and concentrated under reduced pressure. A colorless oil of molecule A14 is obtained after purification by flash chromatography (eluent: cyclohexane, AcOEt).
Yield: 96.61 g (91%)

¹H NMR. (CDCl₃, ppm): 0.87 (3H); 1.26 (12H); 1.65 (2H); 2.02 (3H); 2.34 (2H); 2.41 (1H); 3.48 (1H); 3.56 (1H); 4.58 (1H).
LC/MS (ESI): 270.2; (calculated ([M+H]⁺): 270.2).

### Molecule A15: Product Obtained by the Reaction Between Molecule A14 and N-Boc-ethylenediamine.

By a method similar to the one used for the preparation of molecule A10 applied to molecule A14 (30.00 g, 111.36 mmol) and to BocEDA (21.41 g, 133.64 mmol), a white solid is obtained after recrystallization in acetonitrile.
Yield: 34.90 g (76%)

¹H NMR (CDCl₃, ppn): 0.88 (3H); 1.10-1.70 (14H); 1.43 (9H); 1.80-1.91 (1H); 1.92-2.01 (1H); 2.04-2.42 (4H); 3.13-3.70 (6H); 4.27-4.31 (0.15H); 4.47-4.53 (0.85H); 4.83 (0.15H); 5.02 (0.85H); 6. 85 (0.15H); 7.21 (0.85H),

LC/MS (ESI): 412.2; (calculated ([M+H]⁺: 412.3).

### Molecule AA7

By a method similar to the one used for the preparation of molecule AA1 applied to molecule A15 (34.90 g, 84.79 mmol), a white solid of molecule AA7 in hydrochloride salt form is obtained after solubilization in a DCM/acetonitrile mixture and concentration under reduced pressure.
Yield: 29.50 g (99%)

¹H NMR (DMSO-d6, ppm): 0.85 (3H); 1.07-1.61 (14H); 1.70-2.06 (4H); 2.10-2.35 (2H); 2.76-2.87 (2H); 3.24-3.47 (3.25H); 3.56-3.64 (0.75H); 4.13-4.19 (0.75H); 4.31-4.36 (0.25H): 8.05-8.36 (3.75H); 8.50 (0.25H).
LC/MS (ESI): 312.2; (calculated ([M+H]⁺): 312.3.

### Example AA8

### Molecule AA8

Molecule AA8 is obtained by the conventional method of peptide synthesis in a solid phase (SPPS) on 2-chlorotrityl resin.

A solution of ethylenediamine (FDA, 30.48 mL, 456 mmol) in DCM (200 mL) is poured onto the 2-chlorotrityl resin (20 g, 1.24 mmol/g) previously washed with DCM in a reactor suitable for SPPS. After 2 h of stirring at room temperature, methanol (0.8 mL/g, 3.2 mL) is added and the mixture is stirred for 45 min. The resin is filtered, washed successively with DCM, DMF, DCM, isopropanol and DCM. The protected amino acids N-Fmoc-L-glycine and N-Fmoc-L-proline, then palmitic acid (3 equivalents) are coupled successively using 1-[bis(dimethylamino)methylene]-1 H-1,2,3 -triazolo[4,5-b]pyridinium 3-oxide hexafluorophosphate (HATU, 3 equivalents) as coupling agent in the presence of DIPEA (6 equivalents) in DMF. A 20% piperidine solution in DMF is used for the steps of cleavage of the protective Fmoc group. The resin is washed with DMF, isopropanol and DCM after each coupling and deprotection step. The cleavage of the product from the resin is carried out using a 1: 1 TFA/DCM mixture. The solvents are evaporated under reduced pressure, the residue is solubilized in DCM (500 mL), and the organic phase is washed with an aqueous 1 N NaOH solution (200 mL), then a saturated NaCl solution (2×200 mL). After drying on Na₂SO₄, the organic phase is filtered, concentrated under reduced pressure, and the residue is triturated in IPE (200 mL), then dried at reduced pressure.
Yield: 9.19 g (89%)

¹H NMR (MeOD-d4, ppm): 0.90 (3H); 1.22-1.43 (24H); 1.55-1.67 (2H); 1.91-2.04 (2H); 2.04-2.15 (1H); 2.17-2.29 (1H); 2.39 (2H); 2.69-2.82 (2H); 3.25-3.36 (2H); 3.58-3.70 (2H); 3.70-3.97 (2H); 4.25-4.34 (0.9H); 4.44-4.50 (0.1H).
LC/MS (ESI): 453.3; (calculated ([M+H]⁺): 453.4).

### Example AA9

### molecule AA9

By a SPPS method similar to the one used for the preparation of molecule AA8 and applied to the 2-chlorotrityl resin (25 g, 1.1.4 mmol/g), EDA (285 mmol), N-Fmoc-L-Leueine (3 equivalents) and molecule A9 (3 equivalents), a white solid of molecule AA9 is obtained.
Yield: 11.34 g (83%)

¹H NMR (MeOD-d4, ppm): 0.87-1.00 (9H); 1.23-1.41 (20H); 1.51-1.73 (5H); 1.83-2.44 (6H); 2.69-2.81 (2H); 3.17-3.34 (2H); 3.43-3.70 (2H); 4.28-4.39 (1.8 H); 4.45-4.48 (0.2H).
LC/MS (ESI): 481.4; (calculated ([M+H]⁺): 481.4).

AB: Synthesis of the Co-Polyamino Acids

Statistical co-polyamino acids according to formula VII or VIIa

TABLE 1b
List of the co-polyamino acids synthesized according to the invention

| | CO-POLYAMINOACIDS BEARING CARBOXYLATE |
|---|---|
| No. | CHARGES AND HYDROPHOBIC RADICALS |
| AB1 | 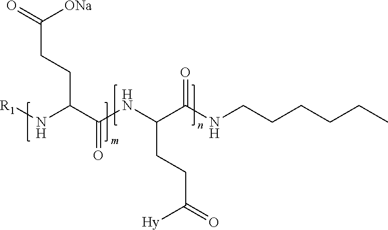 |
| | i = 0.05, DP (m + n) = 23 |
| | 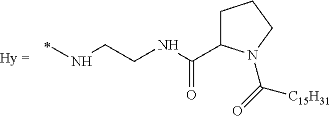 |
| | R₁ = H or pyroglutamate |
| AB2 | 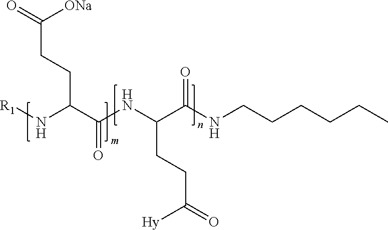 |
| | i = 0.05, DP (m + n) = 35) |
| | 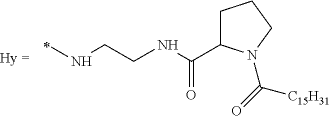 |
| | R₁ = H or pyroglutamate |
| AB3 | 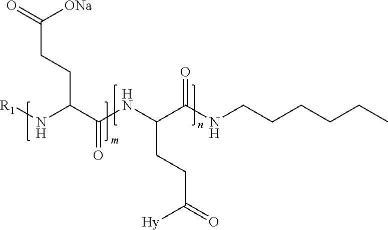 |
| | i = 0.10, DP (m + n) = 35 |
| | 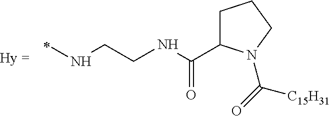 |
| | R₁ = H or pyroglutamate |
| AB4 | 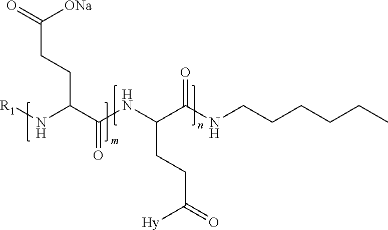 |
| | i = 0.052, DP (m + n) = 35 |
| | 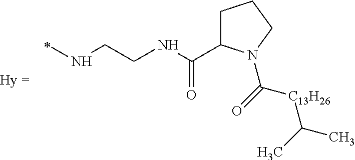 |
| | R₁ = H or pyroglutamate |
| AB5 | 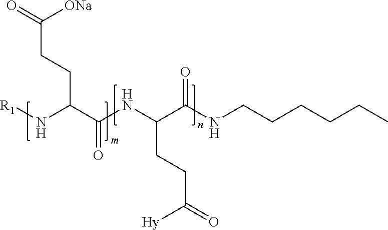 |
| | i = 0.05, DP (m + n) = 23 |
| | 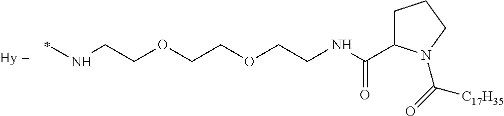 |
| | R₁ = H or pyroglutamate |
| AB11 | 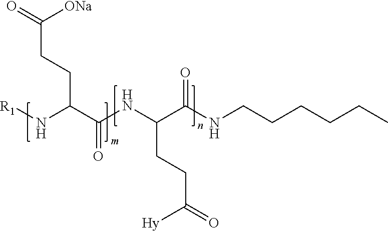 |
| | i = 0.16, DP (m + n) = 38 |
| | 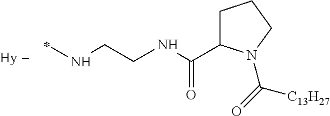 |
| | R₁ = CH₃CO or pyroglutamate |
| AB12 | 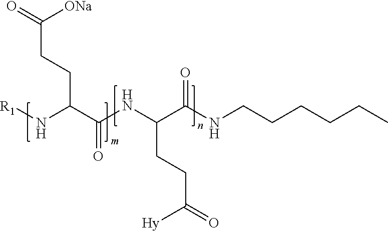 |
| | i = 0.10, DP (m + n) = 60) |
| | 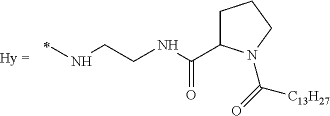 |
| | R₁ = pyroglutamate |
| AB13 | 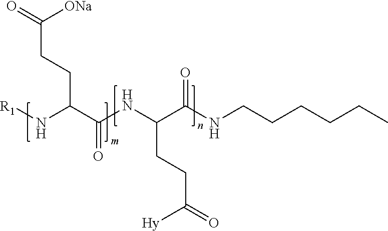 |
| | i = 0.15, DP (m + n) = 39 |
| | 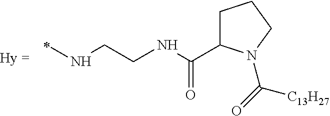 |
| | R₁ = H or pyroglutamate |
| AB14 | 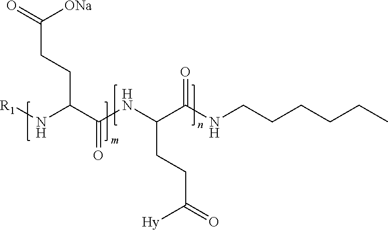 |
| | i = 0.20, DP (m + n) = 39 |
| | 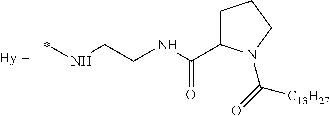 |
| | R₁ = pyroglutamate |
| AB15 | 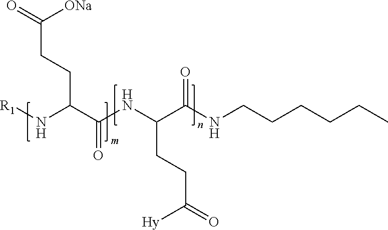 |
| | i = 0.21, DP (m + n) = 22 |
| | 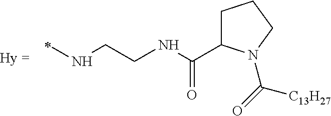 |
| | R₁ = pyroglutamate |
| AB16 | 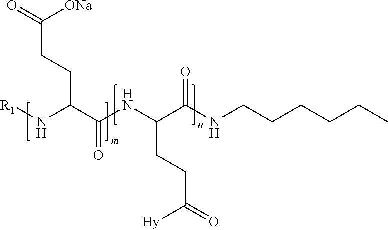 |
| | i = 0.15, DP (m + n) = 39 |
| | Hy |
| | 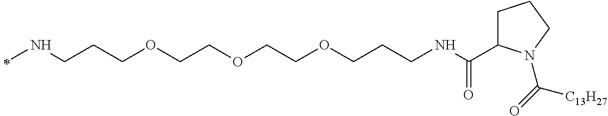 |
| | R₁ = pyroglutamate |
| AB17 | 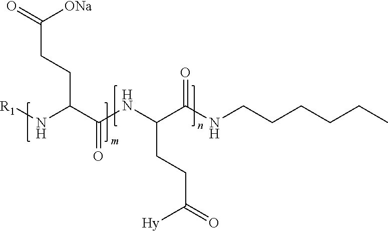 |
| | i = 0.15, DP (m + n) = 39 |
| | 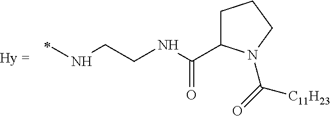 |
| | R₁ = pyroglutamate |
| AB18 | 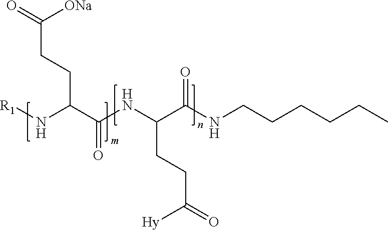 |
| | i = 0.15, DP (m + n) = 40 |
| | 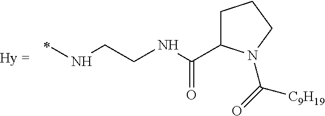 |
| | R₁ = pyroglutamate |
| AB19 | 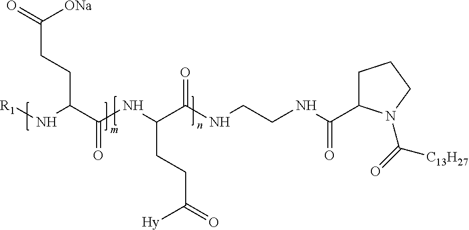 |
| | i = 0.125, DP (m + n) = 40 |
| | 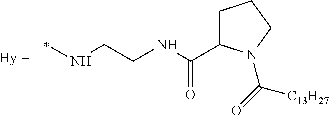 |
| | R₁ = pyroglutamate |
| AB20 | 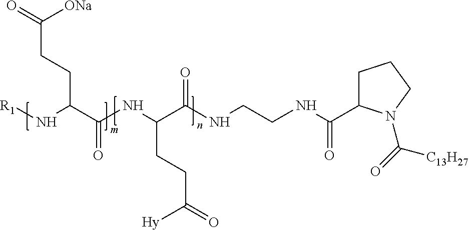 |
| | i = 0.175, DP (m + n) = 40 |
| | 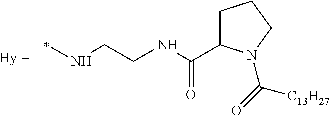 |
| | R₁ = pyroglutamate |
| AB22 | 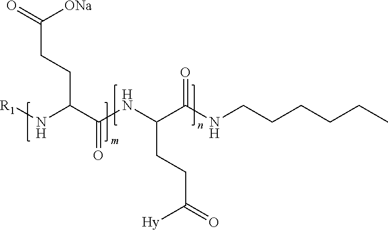 |
| | i = 0.15, DP (m + n) = 38 |
| | 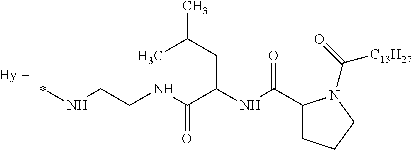 |
| | R₁ = pyroglutamate |

Co-Polyamino Adds According to formula VII or VIIb:

TABLE 1c
list of the co-polyamino acids synthesized according to the invention.

| | CO-POLYAMINOACIDS BEARING CARBOXYLATE |
|---|---|
| No. | CHARGES AND HYDROPHOBIC RADICALS |
| AB6 | 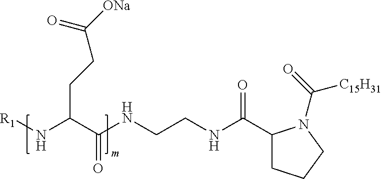 |
| | i = 0.04, DP (m) = 25 |
| | R₁ = H or pyroglutamate |
| AB7 | 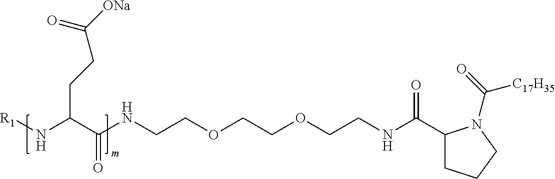 |
| | i = 0.033, DP (m) = 30 |
| | R₁ = H or pyroglutamate |
| AB8 | 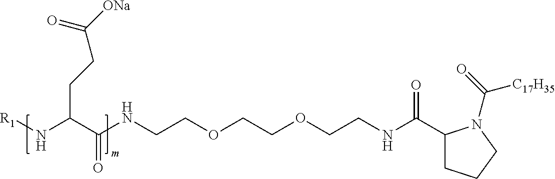 |
| | i = 0.021, DP (m) = 48 |
| | R₁ = H or pyroglutamate |
| AB9 | 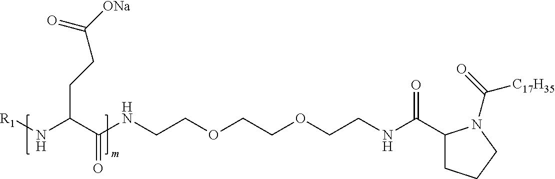 |
| | i = 0.015, DP (m) = 65 |
| | R₁ = H or pyroglutamate |
| AB10 | 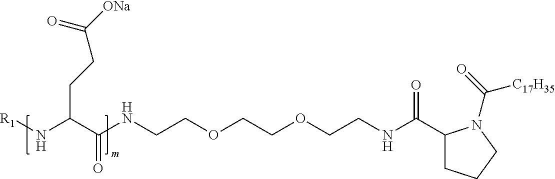 |
| | i = 0.017, DP (m) = 60 |
| | R₁ = CH₃—CO—, H or pyroglutamate |
| AB21 | 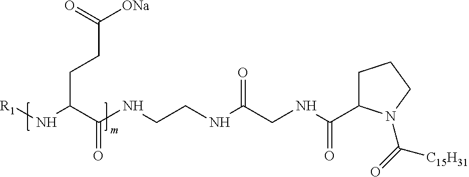 |
| | i = 0.042, DP (m) = 24 |
| | R₁ = H or pyroglutamate |

### Part AB: Synthesis of the Co-Polyamino Acids

### Example AB1

### Co-Polyamino Acid AB1—Sodium Poly-L-Glutamate Modified by Molecule AA1 and having a Number Average Molecular Weight (Mn) of 2900 g/mol

Co-polyamino acid AB1-1: poly-L-glutamic acid having a relative number average molecular weight (Mn) of 3861 g/mol resulting from the polymerization of γ-benzyl-L-glutamate N-carboxyanhydride initiated by hexylamine

In an oven-dried round-bottom flask, γ-benzyl-L-glutamate N-carboxyanhydride (89.9 g, 341 mmol) is placed for 30 min under vacuum, then anhydrous DMF (200 mL) is added. The mixture is then stirred under argon until the dissolution is complete, cooled to 4° C., then hexylamine (2.05 mL, 15.5 mmol) is added rapidly. The mixture is stirred at from 4° C. to room temperature for 2 days. The reaction mixture is then heated at 65° C. for 2 h, cooled to room temperature, then poured dropwise into diisopropyl ether (3 L) under stirring. The white precipitate is recovered by filtration, washed with diisopropyl ether (2×200 mL), then dried under vacuum at 30° C. to yield a poly(gamma-benzyl-L-glutamic) acid (PBLG).

A 33% hydrobromic acid (HBr) solution in acetic acid (240 mL, 1.37 mol) is added dropwise to a solution of PBLG (74.8 g) in trifluoroacetic acid (TFA, 340 mL) at 4° C. The mixture is stirred at room temperature for 2 h, then poured dropwise onto a 1:1 (v/v) mixture of diisopropyl ether and water under stirring (4 L). After 2 h of stirring, the heterogeneous mixture is left to rest overnight. The white precipitate is recovered by filtration, washed with a 1:1 (v/v) mixture of diisopropyl ether and water (340 mL), then with water (340 mL).

The solid obtained is then solubilized in water (1.5 L) by adjusting the pH to 7 by addition of a 10 N aqueous sodium hydroxide solution, then a 1 N aqueous sodium hydroxide solution. After solubilization, the theoretical concentration is adjusted to 20 g/L theoretical by addition of water until obtaining a final volume of 2.1 L.

The solution is filtered through a 0.45 μm filter, then purified by ultrafiltration against a 0.9% NaCl solution, then water until the conductimetry of the permeate is less than 50 μS/cm. The solution of co-polyamino acid is then concentrated until a final volume of 1.8 L is obtained.

The aqueous solution is then acidified by adding 37% hydrochloric acid solution until a pH of 2 is reached. After 4 h of stirring, the precipitate obtained is filtered, washed with water (2×340 mL), then dried under vacuum at 30° C. to yield a poly-L-glutamic acid having a number average molecular weight (Mn) of 3500 g/mol with respect to a polyoxyethylene (PEG) standard.

### Co-Polyamino Acid AB1

The co-polyamino acid AB-1 (10.0 g) is solubilized ire DMF (700 mL) at 30-40° C., then cooled to 0° C. Molecule AA1 in hydrochloride salt form (1.64 g, 3.8 mmol) is suspended in DMF (23 mL) and triethylamine (0.39 g, 3.8 mmol) is then added and the mixture is heated slightly under stirring until the dissolution is complete. The NMM (7.6 g, 75 mmol) in DMF (14 mL) and ethyl chloroformate (ECF, 8.2 g, 75 mmol) are added to the solution of co-polyamino acid at 0° C. After 10 min at 0° C., the solution containing molecule AA1 is added and the medium is maintained at 30° C. for 2 h. The reaction medium is poured dropwise under stirring onto 5.5 L of water containing 15%(w/w) NaCl and HCl (pH 2), then allowed to rest overnight. The precipitate is collected by filtration and dried under a vacuum for approximately 30 min. The white solid obtained is solubilized in water (500 mL) and the pH is adjusted to 7 by slow addition of a 1 N NaOH aqueous solution. After filtration through a 0.45 μm filter, the clear solution obtained is purified by ultrafiltration against a 0.9% NaCl solution, then water until the conductimetry of the permeate is less than 50 μS/cm. After unloading, the solution is filtered through a 0.2 μm filter and stored at 2-8° C.
Dry extract: 24.9 mg/g

A mean polymerization degree (PD) of 23 is estimated by ¹H NMR in D2O by comparing the integration of the signals resulting from the grafted hydrophobe with that of the signals resulting from the main chain.
According to 1H NMR: i=0.05

The calculated average molecular weight of the co-polyamino acid AB1 is calculated based on the molecular weights of the radicals R₁ and R₂, of the aspartate and/or glutamate residues (including an amide bond), of the hydrophobic radical, of DS and of DP.
The calculated average molecular weight of the co-polyamino acid AB1 is 3945 g/mol.
HPLC-aqueous SEC (calibrant PEG): Mn=2900 g/mol.

### Example AB2

### Co-Polyamino Acid AB2—Sodium Poly-L-Glutamate Modified by Molecule AA1 and having a Number Average Molecular Weight (Mn) of 3700 g/mol

By a method similar o the one used for the preparation of the co-polyamino acid AB1 applied to the hydrochloride salt of molecule AA1 (1.64 g, 3.8 mmol) and to a poly-L-glutamic acid having a relative Mn of 5200 g/mol (10.0 g) obtained by a method similar to the one used for the preparation of the co-polyamino acid AB1-1, a sodium poly-L-glutamate modified by molecule AA1 is obtained.
Dry extract: 14.1 mg/g
DP (estimated based on ¹H NMR): 35
Based on ¹H NMR: i=0.05
The calculated average molecular weight of the co-polyamino acid AB2 is 5972 g/mol.
HPLC-aqueous SEC (calibrant PEG): Mn=3700 g/mol.

### Example AB3

### Co-Polyamino acid AB3—Sodium Poly-L-Glutamate Modified by Molecule AA1 and having a Number Average Molecular Weight (Mn) of 4900 g/mol

By a method similar to the one used for the preparation of the co-polyamino acid AB1 applied to the hydrochloride salt of molecule AA1 (3.30 g, 7.6 mmol) and to a poly-L-glutamic acid having a relative number average molecular weight (Mn) of 5200 g/mol (1.0.0 g) obtained by a method similar to the one used for the preparation of the co-polyamino acid AB1-1, a sodium poly-L-glutamate modified by molecule AA1 is obtained.

Dry extract: 23.4 mg/g
DP (estimated based on NMR): 35
The calculated average molecular weight of the co-polyamino acid AB3 is 6594 g/mol.
Based on ¹H NMR: i=010
HPLC-aqueous SEC (calibrant PEG): Mn=4900 g/mol.

### Example AB4

### Co-Polyamino Acid AB4—Sodium Poly-L-Glutamate Modified by Molecule AA2 and having a Number Average Molecular Weight (Mn) of 1800 g/mol

By a method similar to the one used for the preparation of the co-polyamino acid AB1 applied to the hydrochloride salt of molecule AA2 (1.09 g, 2.4 mmol) and to a poly-L-glutamic acid having an average molecular weight Mn=5600 g/mol (6.3 g) obtained by a method similar to the one used for the preparation of the co-polyamino acid AB1-1, but with a step of deprotection of the benzyl esters using trimethylsilane iodide according to the protocol described in the publication J, Am. Chem. Soc. 2000, 122, 26-34 (Subramanian G., et al.), a sodium poly-L-glutamate modified by molecule AA2 is obtained.
Dry extract: 21.5 mg/g
DP (estimated based on ¹H NMR): 35
Based on ¹H NMR: i=0.052
The calculated average molecular weight of of the co-polyamino acid AB4 is 6022 g/mol.
HPLC-aqueous SEC (calibrant PEG): Mn 1800 g/mol.

### Example AB5

### Co-Polyamino acid AB5—Sodium Poly-L-Glutamate Modified by Molecule AA3 and having a Number Average Molecular Weight (Mn) of 2600 g/mol

By a method similar to the one used for the preparation of the co-polyamino acid AB1 applied to the hydrochloride salt of molecule AA3 (2.06 g, 3.8 mmol) and to a poly-L-glutamic acid (9.8 g) obtained by a method similar to the one used for the preparation of the co-polyamino acid AB1-1, a sodium poly-L-glutamate modified by molecule AA3 is obtained.
Dry extract: 20.9 mg/g
DP (estimated based on ¹H NMR): 23
Based on ¹H NMR: i=0.05
The calculated average molecular weight of the co-polyamino acid ABS is 4079 g/mol.
HPLC-aqueous SEC (calibrant PEG): Mn=2600 g/mol.

### Example AB11

### Co-Polyamino Acid AB11—Sodium poly-L-Glutamate Capped at one of its Extremities with an Acetyl Group and Modified by Molecule AA4 and having a Number Average Molecular Weight (Mu) of 4000 g/mol

Copolyamino acid AB1 1-1: poly-L-glutamic acid resulting from the polymerization of γ-benzyl-L-glutamate N-carboxyanhydride initiated by hexylamine and capped at one of its extremities by an acetyl group.

In an oven-dried round-bottom flask, γ-benzyl-L-glutamate N-carboxyanhydride (200.0 g, 760 mmol) is placed under a vacuum for 30 min, then anhydrous DMF (450 mL) is introduced. The mixture is then stirred under argon until the dissolution is complete, cooled to 4° C., then hexylamine (2.64 mL 20.0 mmol) is introduced rapidly. The mixture is stirred at from 4° C. to room temperature for 2 days, then poured dropwise into diisopropyl ether (6.7 L) under stirring. The white precipitate is recovered by filtration, washed with diisopropyl ether (IPE, 2×450 mL) then dried under reduced pressure at 30° C. to give a poly(gamma-benzyl-L-glutamic) acid (PBLG).

The PBLG obtained is solubilized in THF (900 mL), then DIPEA (35 mL, 200 mmol) and acetic anhydride (18.9 mL, 200 mmol) are added successively. After 16 h of stirring at room temperature, the reaction mixture is poured into IPE (5.4 L) under stirring. The white precipitate is recovered by filtration, washed with diisopropyl ether (IPE, 2×450 mL) and dried under reduced pressure at 30° C. to yield a PBLG capped at one of its extremity with an acetyl group (PBLG-Ac).

A 33% hydrobromic acid (HBr) solution in acetic acid (255 L, 1.44 mol) is added dropwise to a solution of PBLG-AC (80.0 g) in trifluoroacetic acid (TFA, 360 mL) at 4° C. The mixture is stirred at room temperature for 2 h, then poured dropwise onto a 1:1 (v/v) mixture of diisopropyl ether and water under stirring (4.3 L). After 2 h of stirring, the heterogeneous mixture is let to rest overnight. The white precipitate is recovered by filtration, washed with a 1:1 (v/v) mixture of diisopropyl ether and water (360 mL), then with water (360 mL).

The solid obtained is then dissolved in water (1.5 L) by adjusting the pH to 7 by adding a 10 N aqueous sodium hydroxide solution, then a 1 N aqueous sodium hydroxide solution. After solubilization, the theoretical concentration is adjusted to 20 g/L theoretical by addition of water to obtain a final volume of 2.4 L.

The solution is filtered through a 0.45 μm filter, then purified by ultrafiltration against a 0.9% NaCl solution, then water until the conductimetry of the permeate is less than 50 μS/cm. The co-polyamino acid solution is then concentrated until a final volume of 1.8 L is obtained.

The aqueous solution is then acidified by addition of 1 N hydrochloric acid solution until a pH of 2 is reached. After stirring overnight, the precipitate obtained is filtered, washed with water (360 mL), then dried under a vacuum at 30° C. to yield a poly-L-glutamic acid capped at one of its extremities with an acetyl group.

### Co-Polyamino Acid AB11

The hydrochloride salt of molecule AA4 (4.56 g, 11.29 mmol) is dissolved in chloroform (60 mL), and triethylamine (1.14 g, 11.29 mmol) is added NMM (7.6 g, 75.26 mmol) and then 2-hydroxypyridine-N-oxide (HOPO) (2.51 g, 22.58 mmol) are added successively to a solution of co-polyamino acid AB11-1 (10.0 g, 75.3 mmol) in DMF (420 mL). The reaction medium is then cooled to 0° C., then EDC (4.33 g, 22.58 mmol) is added, the mixture is stirred for 1 h at 0° C. then the solution of molecule AA4 is added. The reaction medium is stirred for 2 h at from 0° C. to room temperature. The reaction medium is filtered through a 0.2-mm woven filter and poured dropwise onto 3.95 L of water containing 15% (w/w) NaCl and HCl (pH 2) under stirring. At the end of the addition, the pH is readjusted to 2 with a 37% HCl solution, and the suspension is let to rest overnight. The precipitate is collected by filtration, then solubilized in 780 mL of water by slow addition of a IN aqueous NaOH solution until the pH is 7 under stirring. After filtration through a 0.45 μm filter, the solution is diluted by addition of water, then acetone is added to obtain a solution containing 30% iw) acetone. This solution is filtered through an activated charcoal filter, then the acetone is distilled (40° C. 100 mbar). After filtration through a 0.45 μm filter, the product is purified by ultrafiltration against a 0.9% aqueous NaCl solution, a carbonate buffer solution (150 mM), a 0.9% aqueous NaCl solution, a phosphate buffer solution (150 mM), a 0.9% aqueous NaCl solution, then water until the conductimetry of the permeate is less than 50 μS/cm. The solution is then concentrated, filtered through a 0.2 μm filter and stored at 2-8° C.

Dry extract: 19.7 mg/g

DP (estimated based on ¹H NMR): 38

Based on ¹H NMR: 0.16

The calculated average molecular weight of the co-polyamino acid AB11is 7877 g/mol.

HPLC-organic SEC (calibrant PEG): Mn=4000 g/mol.

### Example AB12

### Co-Polyamino Acid AB12—Sodium Poly-L-Glutamate Modified by Molecule AA4 and having a Number Average Molecular Weight (Mn) of 7600 g/mol

Co-polyamino acid AB 12-1: poly-L-glutamic resulting from the polymerization of γ-benzyl-L-glutamate N-carboxyanhydride initiated by hexylamine and capped at one of its extremities with a pyroglutamate group.

A poly-L-glutamic acid (20.0 g) obtained by a method similar to the one used for the preparation of the co-polyamino acid AB1-1 is solubilized in DMF at 80° C., and then maintained at this temperature. After 24 h, the reaction medium is poured into a 15% (w/w) NaCl solution and at pH 2. After 4 h, the white solid is collected by filtration, rinsed with water, then dried under vacuum at 30° C.

### Co-Polyamino Acid AB12

By a similar method to the one used for the preparation of co-polyamino acid AB11 applied to the hydrochloride salt of molecule AA4 (2.74 g, 6.79 mmol) and to the co-polyamino acid AB12-1 (9.0 g), a sodium poly-L-glutamate acid modified by molecule AA4 is obtained.
Dry extract: 21.9 mg/g
DP (estimated based on ¹H NMR): 60
Based on ¹H NMR: i=0.1
The calculated average molecular weight of the co-polyamino acid AB12 is 11,034 g/mol.
HPLC-organic SEC (calibrant PEG): Mn=7600 g/mol.

### Example AB13

### Co-Polyamino Acid AB13—Sodium Poly-L-Glutamate Modified by Molecule AA4 and having a Number Average Molecular Weight (Mn) of 4300 g/mol

By a method similar to the one used for the preparation of the co-polyamino acid AB11 applied to the hydrochloride salt of molecule AA4 (5.47 g, 13.55 mmol) and to a poly-L-glutamic acid obtained by a method similar to the one used for the preparation of the co-polyamino acid AB12-1 (12.0 g), a sodium poly-L-glutamate modified by molecule AA4 is obtained.
Dry extract: 22.9 mg/g
DP (estimated based on ¹H NMR): 39
Based on ¹H NMR: i=0.15
The calculated average molecular weight of the co-polyamino acid AB13 is 7870 g/mol.
HPLC-organic SEC (calibrant PEG): Mn 4300 g/mol.

### Example AB14

### Co-Polyamino Acid AB14—Sodium Poly-L-Glutamate Modified by Molecule AA4 and having a Number Average Molecular Weight (Mn) of 4200 g/mol

By a method similar to the one used for the preparation of the co-polyamino acid AB11 applied to the hydrochloride salt of AA4 (7.31 g, 18.1 mmol) and to a poly L-glutamic acid obtained by a method similar to the one used for the preparation of the co-polyamino acid AB12-1 (12.0 g), a sodium poly-L-glutamate modified by molecule AA4 is obtained.
Dry extract: 25.9 mg/g
DP (estimated based on ¹H NMR): 39
Based on ¹H NMR: i=0.2
The calculated average molecular weight of the co-polyamino acid AB14 is 8509 g/mol.
HPLC-organic SEC (calibrant PEG): Mn=4200 g/mol.

### Example AB15: Co-Polyamino Acid AB15—Sodium Poly-L-Glutamate Modified by Molecule AA4 and having a Number Average Molecular Weight (Mn) of 2700 g/mol

By a method similar to the one used for the preparation of the co-polyamino acid AB11 applied to the hydrochloride salt of molecule AA4 (7.29 g, 18.06 mmol) and to a poly-L-glutamic acid obtained by a method similar to the one used for the preparation of the co-polyamino acid AB12-1 (12.0 g), a sodium poly-L-glutamate modified by molecule AA4 is obtained.
Dry extract: 23.9 mg/g
DP (estimated based on ¹H NMR): 22
Based on ¹H NMR: i=0.21
The calculated average molecular weight of the co-polyamino acid AB15 is 4899 g/mol.
HPLC-organic SEC (calibrant PEG): Mn=2700 g/mol.

### Example AB16

### Co-Polyamino Acid AB16—Sodium Poly-L-Glutamate Modified by Molecule AA5 and having a Number Average Molecular Weight (Mn) of 4500 g/mol

By a method similar to the one used for the preparation of the co-polyamino acid AB11 applied to the hydrochloride salt of molecule AA5 (7.74 g, 13.72 mmol) and to a poly-L-glutamic acid obtained by a method similar to the one used for the preparation of the co-polyamino acid AB12-1 (12.0 g), a sodium poly-L-glutamate modified by molecule AA5 is obtained.
Dry extract: 26.8 mg/g
DP (estimated based on ¹H NMR): 39
Based on ¹H NMR: i=0.15
The calculated average molecular weight of the co-polyamino acid AB16 is 8808 g/mol.
HPLC-organic SEC (calibrant PEG): Mn=4500 g/mol.

### Example AB17

### Co-Polyamino Acid AB17—Sodium Poly-L-Glutamate Modified by Molecule AA6 and having a Number Average Molecular Weight (Mn) of 4000 g/mol

By a method similar to the one used for the preparation of the co-polyamino acid AB11 applied to the hydrochloride salt of molecule AA6 (4.66 g, 13.72 mmol) and to a poly-L-glutamic acid obtained by a method similar to the one used for the preparation of the co-polyamino acid AB12-1 (12.0 g), a sodium poly-L-glutamate modified by molecule AA6 is obtained.
Dry extract: 22.9 mg/g
DP (estimated based on ¹H NMR): 39
Based on ¹H NMR: i=0.15
The calculated average molecular weight of the co-polyamino acid AB17 is 7706 g/mol.
HPLC-organic SEC (calibrant PEG): Mn=4000 g/mol.

### Example AB18

### Co-Polyamino acid AB18—Sodium Poly-L-Glutamate Modified by Molecule AA7 and having a Number Average Molecular Weight (Mn) of 4000 g/mol

Co-polyamino acid AB18-1: poly-L-glutamic acid resulting from the polymerization of γ-benzyl-L-glutamate N-carboxyanhydride initiated by hexylamine

In a double-jacket reactor, γ-benzyl-L-glutamate N-carboxyanhydride (500 g, 1.90 mol) is solubilized in anhydrous DMF (1.1 L). The mixture is then stirred until the dissolution is complete, cooled to 0° C., then hexylamine (6.27 mL, 47.5 mmol) is introduced rapidly. The mixture is stirred at 0° C. for 5 h, at from 0° C. to 20° C. for 7 h, then at 20° C. for 7 h. The reaction medium is then heated at 65° C. for 2 h, cooled to 55° C., and methanol (3 L) is introduced over 1 h 30. The reaction mixture is then cooled to 0° C. and stirred for 18 h. The white precipitate is recovered by filtration, washed with diisopropyl ether (2×800 mL), and then dried under reduced pressure at 30° C. to give a poly(gamma-benzyl-L-glutamic) acid (PBLG).

Pd/Al2O3 (36 g) is added under an argon atmosphere to a solution of PBLG (180 g) N,N-dimethylacetamide (DMAc, 450 mL). The mixture is placed under a hydrogen atmosphere (10 bar) and stirred at 60° C. for 24 h. After cooling at room temperature and filtration of the catalyst on a sintered filter P4 then a 0.2 μm Omnipore hydrophilic PTFE membrane, a solution of water at pH 2 (2.7 L) is poured dropwise onto the DMAc solution, over a period of 45 min and under stirring. After 18 h under stirring, the white precipitate is recovered by filtration, washed with water, then dried under reduced pressure at 30° C.

### Co-Polyamino Acid AB18

By a method similar to the one used for the preparation of the co-polyamino acid AB11 applied to the hydrochloride salt of molecule AA7 (1.99 g, 5.72 mmol) and to the co-polyamino acid AB18-1 (5.0 g), a sodium poly-L-glutamate modified by molecule AA7 is obtained.
Dry extract: 16.1 mg/g
DP (estimated based on ¹NMR): 40
Based on ¹H NMR: i=0.15
The calculated average molecular weight of the co-polyamino acid AB18 is 7734 g/mol.

HPLC-organic SEC (calibrant PEG): Mn=4000 g/mol.

### Example AB19

### Co-Polyamino Acid AB19—Sodium Poly-L-Glutamate Modified by Molecule AA4 and having a Number Average Molecular Weight (Mn) of 4300 g/mol

By a method similar to the one used for the preparation of the co-polyamino acid AB18 applied to the hydrochloride salt of molecule AA4 (4.37 g, 10.83 mmol) and to a poly-L-glutamic acid (15.0 g) obtained by a method similar to the one used for the preparation of the co-polyamino acid AB18-1 using molecule AA4 as initiator instead of hexylamine, a sodium poly-L-glutamate modified by molecule AA4 is obtained.
Dry extract: 29.2 mg/g
DP (estimated based on ¹H NMR): 40
Based on ¹H NMR: i=0.125
The calculated average molecular weight of the co-polyamino acid AB19 is 7682 g/mol.
HPLC-organic SEC (calibrant PEG): Mn=4300 g/mol.

### Example AB20

### Co-Polyamino Acid AB20—Sodium Poly-L-Glutamate Modified by Molecule AA4 and having a Number Average Molecular Weight (Mn) of 6300 g/mol

By a method similar to the one used for the preparation of the co-polyamino acid AB18 applied to the hydrochloride salt of molecule AA4 (6.56 g, 16.24 mmol) and to a poly-L-glutamic acid (15.0 g) obtained by a method similar to the one used for the preparation of the co-polyamino acid AB18-1 using molecule AA4 as initiator instead of hexylamin, a sodium poly-L-glutamate modified by molecule AA4 is obtained.
Dry extract: 23.1 mg/g
DP (estimated based on ¹H NMR): 40
Based on ¹H NMR: i=0.175
The calculated average molecular weight of the co-polyamino acid AB20 is 8337 g/mol.
HPLC-organic SEC (calibrant PEG): Mn=6300 g/mol.

### Example AB22

### Co-Polyamino Acid AB22—Sodium Poly-L-Glutamate Modified by Molecule AA9 and having a Number Average Molecular Weight (Mn) of 4600 g/mol

By a method similar to the one used for the preparation of the co-polyamino acid AB11 applied to molecule AA9 (7.15 g, 18.87 mmol) and to a poly-L-glutamic acid obtained by a method similar to the one used for the preparation of the co-polyamino acid AB18-1 (13.0 g), a sodium poly-L-glutamate modified by molecule AA9 is obtained.
Dry extract: 23.3 mg/g
DP (estimated based on ¹H NMR): 38
Based on ¹n NMR: i=0.15
The calculated average molecular weight of the co-polyamino acid AB22 is 8315 g/mol.
HPLC-organic SEC (calibrant PEG): Mn=4600 g/mol.

### Co-Polyamino Acids According to Formula VII or VIIb

### Example AB6

### Co-Polyamino Acid AB6—Sodium Poly-L-Glutamate Modified at one of its Extremities by Molecule AA1 and having a Number Average Molecular Weight (Mn) of 3400 g/mol

The hydrochloride salt of molecule AA1 (2.03 g, 4.70 mmol), chloroform (5 mL), molecular sieve 4 Å (1.3 g) and the ion exchange resin Amberlite IRN 150 (1.3 g) are introduced successively into an appropriate container. After 1 h of stirring on rollers, the medium is filtered and the resin is rinsed with chloroform. The mixture is evaporated, then co-evaporated with toluene. The residue is solubilized in anhydrous DMF (30 mL) to be used directly in the polymerization reaction.

In an oven-dried round-bottom flask, γ-benzyl-L-glutamate N-carboxyanhydride (25.59 g, 97.2 mmol) is placed under a vacuum for 30 min, then anhydrous DMF (140 mL) is introduced. The mixture is stirred under argon until the solubilization is complete, cooled to 4° C., then the solution of molecule AA1 prepared as described above is introduced rapidly. The mixture is stirred 4° C. and room temperature for 2 days, and then heated at 65° C. for 2 h. The reaction mixture is then cooled to room temperature, then poured dropwise in diisopropyl ether (1.7 L) under stirring. The white precipitate is recovered by filtration, washed two times with diisopropyl ether (140 mL), then dried under a vacuum at 30° C. to obtain a white solid. The solid is diluted in TFA (160 mL), and a 33% hydrobromic acid (HBr) solution in acetic acid (62 mL, 354 mmol) is then added dropwise and at 0° C. The solution is stirred for 2 h at room temperature, then poured dropwise on a 1:1 (v/v) mixture of diisopropyl ether/water and under stirring (1.9 L). After 2 h of stirring, the heterogeneous mixture is let to rest overnight. The white precipitate is recovered by filtration, washed successively with a 1:1 (v/v) mixture of diisopropyl ether and water (280 mL), then with water (140 mL). The solid obtained is solubilized in water (530 mL) by adjusting the pH to 7 by addition of a 10 N aqueous sodium hydroxide solution, then a 1 N aqueous sodium hydroxide solution. After solubilization, the theoretical concentration is adjusted to 20 g/L theoretical by addition of water in order to obtain a final volume of 800 mL. The mixture is filtered through a 0.45 μm filter, then purified by ultrafiltration against a 0.9% NaCl solution, then water until the conductimetry of the permeate is less than 50 μS/cm. The solution of co-polyamino acid is then concentrated to approximately 30 g/L theoretical and the pH is adjusted to 7.0. The aqueous solution is filtered through a 0.2 μm filter and stored at 4° C.
Dry extract: 24.1 mg/g
DP (estimated based on ¹H NMR)=25, thus i=0.04
The calculated average molecular weight of the co-polyamino acid AB6 is 4133 g/mol.
HPLC-aqueous SEC (calibrant PEG): Mn=3400 g/mol.

### Example AB7

### Co-Polyamino Acid AB7—Sodium Poly-L-Glutamate Modified at One of its Extremities by Molecule AA3 and having a Number Average Molecular Weight (Mn) of 4100 g/mol

By a method similar to the one used for the preparation of the co-polyamino acid AB6 applied to the hydrochloride salt of molecule AA3 (2.16 g, 3.94 mmol) and to 25.58 g (97.2 mmol) of y-benzyl-L-glutamate N-carboxyanhydride, a sodium poly-L-glutamate modified at one of its extremities by molecule AA3 is obtained.
Dry extract: 45.5 mg/g
DP (estimated based on ¹H NMR)=30, thus i=0.033
The calculated average molecular weight of the co-polyamino acid AB7 is 5005 g/mol.
HPLC-aqueous SEC (calibrant PEG): Mn=4100 g/mol.

### Example AB8

### Co-Polyamino Acid AB8—Sodium Poly-L-Glutamate Modified at one of its Extremities by Molecule AA3 and having a Number Average Molecular Weight (Mn) of 6500 g/mol

By a method similar to the one used for the preparation of the co-polyamino acid AB6 applied to the hydrochloride of molecule AA3 (2.39 g, 4.36 mmol) and to 50.0 g (189 g mmol) of γ-benzyl-L-glutamate N-carboxyanhydride, a sodium poly-L-glutamate modified at one of its extremities by molecule AA3 is obtained.
Dry extract: 28.5 mg/g
DP (estimated based on ¹H NMR)=48, thus i=0.021
The calculated average molecular weight of the co-polyamino acid ABS is 7725 g/mol.
HPLC-aqueous SEC (calibrant PEG): Mn=6500 g/mol.

### Example A139

### Co-Polyamino Acid AB9—Sodium Poly-L-Glutamate Modified at one of its Extremities by Molecule AA3 and having a Number Average Molecular Weight (Mn) of 10,500 g/mol

By a method similar to the one used for the preparation of the co-polyamino acid AB6 applied to the hydrochloride salt of molecule AA3 (1.64 g, 2.99 mmol) and to γ-benzyl-L-glutamate N-carboxyanhydride (49.3 g, 187 mmol), a sodium poly-L-glutamate modified at one of its extremities by molecule AA3 is obtained.
Dry extract: 23.4 mg/g
DP (estimated based on ¹H NMR) 65, thus i=0.015
The calculated average molecular weight of the co-polyamino acid AB9 is 10,293 g/mol.
HPLC-aqueous SEC (calibrant PEG): Mn=0,500 g/mol.

### Example AB10

### Co-Polyamino Acid AB10—Sodium Poly-L-Glutamate Capped at one of its Extremities with an Acetyl Group and Modified at one of its Extremities by Molecule AA3 and having a Number Average Molecular Weight (Mn) of 10,400 g/mol

The hydrochloric acid of molecule AA3 (0.545 g, 1.00 mmol), chloroform (10 mL), molecular sieve 4 Å (3 g) as well as the ion exchange resin Amberlite IRN 1.50 (3 g) are introduced successively into an appropriate container. After 1 h of stirring on rollers, the medium is filtered and the resin is rinsed with chloroform. The mixture is evaporated and then co-evaporated with toluene. The residue is solubilized in anhydrous DMF (10 mL) to be used directly in the polymerization reaction.

γ-Benzyl-L-glutamate N-carboxyanhydride (17.0 g, 64.6 mmol) is placed under vacuum for 30 min in a round-bottom flask dried beforehand in the oven, then anhydrous DMF (30 mL) is added. The mixture is stirred under argon until the solubilization is complete, cooled to 4° C., then the solution of molecule AA3 prepared as described above is introduced rapidly. The mixture is stirred at from 4° C. to room temperature for 2 days, then precipitated in diisopropyl ether (0.6 L). The precipitate is recovered by filtration, washed two times with diisopropyl ether (40 mL), then dried to give a white solid which is dissolved in 80 mL of THF. To this solution, DIPEA (1.7 mL, 9.8 mmol) and then acetic anhydride (0.9 mL, 9.5 mmol) are added successively. After stirring overnight at room temperature, the solution is poured slowly into diisopropyl ether (480 mL) for a duration of 30 min and under stirring. After 1 h of stirring, the precipitate is filtered, washed two times with diisopropyl ether (80 mL), and then dried under a vacuum at 30° C. to give a poly(gamma-benzyl-L-glutamic) acid capped at one of its extremites by an acetyl group and modified at the other one of its extremities by molecule AA3 in the form of a white solid.

The solid is diluted in TTA (65 mL), and a 33% hydrobromic acid (HBr) solution in acetic acid (45 mL, 257.0 mmol) is then added dropwise and at 4° C. The solution is stirred for 2 h at room temperature, and then poured dropwise onto a 1:1 (v/v) mixture of diisopropyl ether/water and under stirring (780 mL). After 2 h of stirring, the heterogeneous mixture is let to rest overnight. The white precipitate is recovered by filtration, washed successively with a 1:1 (v/v) mixture of diisopropyl ether and water (70 mL), then with water (70 mL). The solid obtained is solubilized in water (300 mL) by adjusting the pH to 7 by addition of a 10 N aqueous sodium hydroxide solution, then a 1 N aqueous sodium hydroxide solution. After solubilization, the theoretical consideration is adjusted to 20 g/L theoretical by addition of water in order to obtain a final volume of 440 mL. The mixture is filtered through a 0.45 μm filter, then purified by ultrafiltration against a 0.9% NaCl solution, then water until the conductimetry of the permeate is less than 50 μS/cm. The solution of co-polyamino acid is then concentrated to approximately 30 g/L theoretical and the pH is adjusted to 7.0. The aqueous solution is filtered through a 0.2 μM filter and stored at 4° C.
Dry extract: 21.5 mg/g
DP (estimated by ¹H NMR)=60, thus i=0.017
The calculated average molecular weight of the co-polyamino acid AB10 is 9619 g/mol.
HPLC-aqueous SEC (calibrant PEG): Mn=10,400 g/mol.

### Example AB21

### Co-Polyamino acid AB21—Sodium Poly-L-Glutamate Modified at one of its Extremities by Molecule AA8 and having a Number Average Molecular Weight (Mn) of 2800 g/mol

γ-Benzyl-L-glutamate N-carboxyanhydride (56.3 g, 214 mmol) is solubilized in anhydrous DMF (110 mL) in an oven-dried round-bottom flask. The mixture is cooled to 4° C., then a solution of molecule AA8 in free amine form (4.5 g, 9.73 mmol) in a mixture of chloroform (40 mL) and DMF (50 mL) is introduced rapidly. The mixture is stirred at from 4° C. to room temperature for 18 h, then heated at 65° C. for 2 h. The reaction mixture is then cooled to room temperature and poured dropwise into diisopropyl ether (2.7 L) under stirring. After 2 h of stirring, the white precipitate is recovered by filtration, triturated with methanol (2×500 mL), then dried under reduced pressure at 30° C. to give a white solid. The solid (45.75 g) is solubilized in N,N-dimethylacetamide (DMAc, 140 mL), then Pd/Al₂O₃ (5 g) is added under an argon atmosphere. The mixture is placed under a hydrogen atmosphere (10 bar) and stirred at 60° C. for 24 h. After cooling at room temperature and filtration of the catalyst on a sintered filter P4 and then through a 0.2 μm Omnipore hydrophilic PTFE membrane, a solution of water at pH 2 (1 L) is poured dropwise onto the DMAc solution, over a period of 45 min and under stirring. After 18 h under stirring, the white precipitate is recovered by filtration, washed with water (4×60 mL), then dried under reduced pressure at 30° C. The solid obtained is solubilized in water (940 mL) by adjusting the pH to 7 by addition of a 1 N aqueous sodium hydroxide solution. The pH is then adjusted to pH 12 and the solution is maintained under stirring for 1 h. After neutralization at pH 7, the solution is filtered through a 0.2 μm filter, diluted with ethanol in order to obtain a solution containing 30% (w/w) ethanol, then filtered through an activated charcoal filter (3M R53SLP). The solution obtained is filtered through a 0.45 μm filter and purified by ultrafiltration against a 0.9% NaCl solution, then water until the conductimetry of the permeate is less than 50 μS/cm. The solution of co-polyamino acid is then concentrated to approximately 30 g/L theoretical and the pH is adjusted to 7. The aqueous solution is filtered through a 0.2 μm filter and stored at 4° C.
Dry extract: 26.8 mg/g
DP (estimated by ¹H NMR)=24, thus i=0.042
The calculated average molecular weight of the co-polyamino acid AB21 is 4049 g/mol.
HPLC-aqueous SEC (calibrant PEG): Mn=2800 g/mol.

### Part CE Co-Polyamino Acid Counter Examples

| No. | CO-POLYAMINO ACID COUNTER EXAMPLES |
|---|---|
| CE1 | 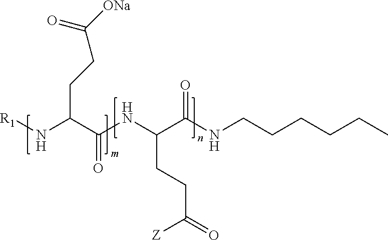 |
| | i = 0.05, DP (m + n) = 22 |
| | 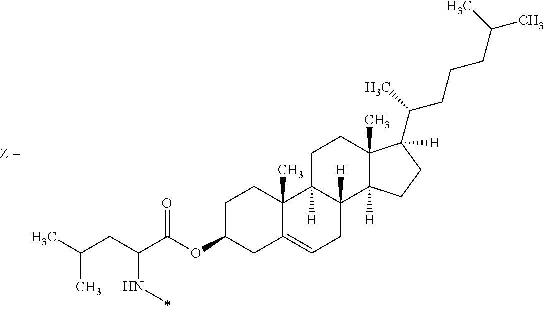 |
| | R₁ = CH₃—CO—, H or pyroglutamate |
| CE2 | 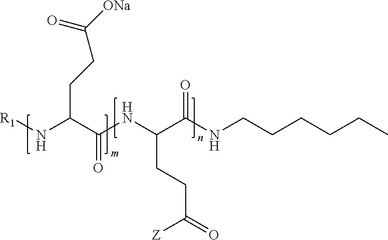 |
| | i = 0.05 DP (m + n) = 43 |
| | 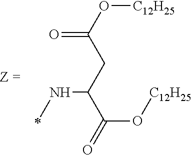 |
| | R₁ = CH₃—CO—, H or pyroglutamate |

The co-polyamino acids CE1 and CE2 are synthesized by a method described in application WO2017211916.

### Part C Compositions

### Example C1

### Solution of Rapid-acting analog insulin lispro at 600 U/mL

This solution is an insulin solution prepared from an insulin lispro powder. This product is a rapid-acting analog insulin. The excipients used are m-cresol, glycerol, zinc chloride, sodium hydroxide and hydrochloric acid for the adjustment of the pH (pH 7-7.8) and water. The concentration of zinc chloride is 1800 μM, the concentration of m-cresol is 35 mM and the concentration of glycerol is 230 mM.

### Example C2

### Insulin glargine Solution at 100-325 U/mL

This solution is an insulin glargine solution prepared from an insulin glargine powder. This product is a slow-acting analog insulin. The excipients used are zinc chloride, m-cresol, glycerol, sodium hydroxide and hydrochloric acid for the adjustment of the (pH 4) and water. The concentration of zinc is 4.5 μM for 1 U/mL of insulin. The concentrations of glycerol and of m-cresol vary depending on the desired concentrations in the final preparations CB1.

### Part CA—Compositions Including Glargine Insulin

Preparation method CA1: Preparation of a concentrated composition of co-polyamino acid/insulin glargine, according to a method using insulin glargine in liquid form (in solution) and a co-polyamino acid in liquid form (in solution).

A stock solution of co-polyamino acid at 7.0-7.5 is added to a solution of insulin glargine described in example C2. A turbidity appears. The pH is adjusted to pH 7.5 by addition of concentrated NaOH and the solution is placed under static conditions at +40° C. until the solubilization is complete. The solution obtained is visually clear and allowed to cool to 20-25° C.

According to the preparation method CA1, compositions of co-polyamino acid/insulin glargine are prepared with concentrations of insulin glargine from 50 U/mL to 200 U/mL.

### Part CB—Compositions Including Insulin Glargine and Insulin Lispro

Preparation method CB1: Preparation of a composition of co-polyamino acid/insulin glargine/insulin lispro at pH 7.2

A solution of lispro described in example C1 and if needed water are added to the concentrated composition of co-polyamino acid/insulin glargine described in example CA1. The pH is adjusted to 7.2 by addition of a concentrated hydrochloric acid solution.

The compositions are filtered through a 0.22 μm filter and stored at 4° C.

### Example CB1

### Compositions of Co-Polyamino Acid/Insulin glargine/insulin Lispro a pH 7.2

According to the preparation method CB1, compositions of co-polyamino acid/insulin glargine/insulin lispro were prepared with insulin glargine concentrations of 75 U/mL or 150 U/mL and insulin lispro concentrations of 25 mL or 50 U/mL. These solutions contain 35 mM m-cresol, 230 mM of glycerol and variable contents of zinc and co-polyamino acids. These compositions are described in Table 1.

TABLE 1
Compositions of insulin glargine and of insulin lispro in the
presence of co-polyamino acid.

| | Co- | | | | | |
|---|---|---|---|---|---|---|
| | poly- | Concentration of | Insulin | Insulin | | Visual |
| Comp- | amino | copolyamino acid | glargine | Lispro | [ZnCl₂] | appear- |
| osition | acid | (mg/mL) | (U/mL) | (U/mL) | (mM) | ance |
| CB1-1 | AB13 | 5 | 150 | 50 | 1 | Clear |
| CB1-2 | | 2.5 | 75 | 25 | 0.5 | Clear |
| CB1-3 | AB17 | 3.8 | 150 | 50 | 1 | Clear |
| CB1-4 | | 1.9 | 75 | 25 | 0.5 | Clear |
| CB1-5 | CE1 | 2.0 | 75 | 75 | 0.5 | Clear |
| CB1-6 | CE2 | 2.0 | 75 | 25 | 0.5 | Clear |

### Part CC: Demonstration of the Physical Stability of the Compositions According to the Invention by the Study of the Previously Prepared Compositions.

Protocol CC1: Study of the physical stability of the compositions of co-polyamino acid/insulin glargine/insulin lispro at pH 7.2.

At least 5 3 mL vials filled with 1 mL of composition of co-polyamino acid insulin glargine/prandial insulin are placed in an oven at 30° C. under static conditions. The vials are inspected visually twice a month in order to detect the appearance of visible particles or turbidity. This inspection is carried out according to the recommendations of the European Pharmacopoeia (EP 2.9.20): the vials are subjected to illumination of at least 2000 lux and are observed on a white background and on a black background. The number of weeks of stability corresponds to the the time from which most of the vials show visible particles or are turbid. The results are described in Table 2.

TABLE 2
Physical stability of the compositions
including AB13 at different concentrations.

| | | Concentration of | |
|---|---|---|---|
| | | co-polyamino acid | Stability |
| Composition | Co-polyamino acid | (mg/mL) | (week) |
| CB1-1 | AB13 | 5 | >14 |
| CB1-2 | | 2.5 | >23 |

### Example CD1

### Precipitation of Insulin Glargine after Mixing of the Compositions of Co-Polyamino Acid/Insulin Glargine 75 U/mL/Insulin Lispro 25 U/mL in a Simulated Physiological Medium

This test demonstrates the precipitation of insulin glargine during the injection into a simulated physiological medium at physiological pH and ionic strength and containing albumin. These conditions make it possible to mimic the behavior of the composition during the subcutaneous injection, 100 μL of a bovine albumin solution at 20 mg/mL in a phosphate buffer at pH 7.4 are added to 100 μL of a composition of co-polyamino acid/insulin glargine 75 U/mL/insulin lispro 25 U/mL. The phosphate buffer (PBS or Phosphate buffer saline) is concentrated in such a manner that the phosphate and NaCl contents are 140 mM and 10 mM, respectively, after mixing the composition. The precipitation of glargine in this medium is monitored at room temperature (20-25° C.) by absorbance measurements at 450 nm of the mixtures for 30 min. The absorbance measurements are carried out using a UV-visible reader of multiwell plates.

The absorbance increases until a plateau is reached. The glargine precipitation time is defined as the time necessary for the measured absorbance to be greater than or equal to 80% of the value of the plateau.

TABLE 3
Precipitation time of insulin glargine after mixing of the compositions
of co-polyamino acid/insulin glargine/insulin lispro with a
medium which simulates the
subcutaneous medium. The composition CB1-2
leads to a very rapid precipitation of glargine after
mixing with a medium which stimulates the sub-cutaneous medium.

| | | Concentration of co- | Precipitation |
|---|---|---|---|
| | Co-polyamino | polyamino acid | time |
| Composition | acid | in mg/mL) | (minutes) |
| CB1-2 | AB13 | 2.5 | 0.5 |
| CB1-5 | CE1 | 2.0 | 4 |
| CB1-6 | CE2 | 2.0 | 4 |

1. A composition in the form of an injectable aqueous solution, whose pH is from 6.0 to 8.0, comprising at least:
a) one basal insulin the isoelectric point (pI) of which is from 5.8 to 8.5,
b) a co-polyamino acid bearing carboxylase charges and hydrophobic radicals Hy, said co-polyamino acid consisting of glutamic or aspartic units, and said hydrophobic radicals Hy being according to the following formula 1:
*(GpR)ᵣ(GpA)ₐ(GpC)ₚ Formula 1
in which
GpR is a radical according to formula II or II′: 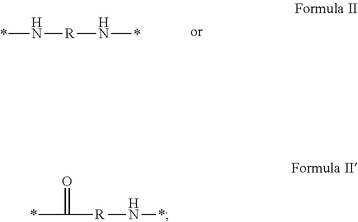
GpA is a radical according to formula III or III′: 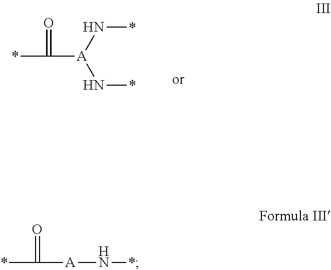
GpC is a radical according to formula IV: 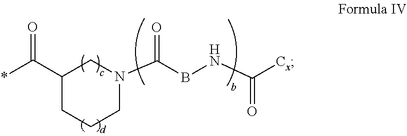
wherein Hy comprises from 15 to less than 30 carbon atoms,
the * indicates the sites of attachment of the different groups bound by amide functions;
a is an integer equal to 0 or 1;
b is an integer equal to 0 or 1;
p is an integer equal to 1 or 2, and
if p is equal to 1, then a is equal to 0 or 1 and GpA is a radical according to formula III′, and
if p is equal to 2, then a is equal to 1 and GpA is a radical according to formula III;
c is an integer equal to 0 or 1, and, if c is equal to 0, then d is equal to 1 or 2;
d is an integer equal to 0, to 1 or 2;
r is an integer equal to 0 or 1, and
if r is equal to 0, then the hydrophobic radical according to formula I is bound to the co-polyamino acid via a covalent bond between a carbonyl of the hydrophobic radical and a nitrogen atom in N-terminal position of the co-polyamino acid, thus forming an amide function, and
if r is equal to 1, then the hydrophobic radical according to formula I is bound to the co-polyamino acid:
via a covalent bond between a nitrogen atom of the hydrophobic radical and a carbonyl of the co-polyamino acid, thus forming an amide function, or
via a covalent bond between a carbonyl of the hydrophobic radical and a nitrogen atom in N-terminal position of the co-polyamino acid, thus forming an amide function;
R is a radical chosen from the group consisting of:
a linear or branched divalent alkyl radical comprising, if GpR is a radical according to formula II, from 2 to 12 carbon atoms, or, if GpR is a radical according to formula II′, from I to 11 carbon atoms;
a linear or branched divalent alkyl radical comprising, if GpR is a radical according to formula II, from 2 to 11 carbon atoms, or, if GpR is a radical according to formula II′, from 1 to 11 carbon atoms, said alkyl radical bearing one or more —CONH₂ functions, and
an unsubstituted ether or polyether radical comprising from 4 to 14 carbon atoms and from 1 to 5 oxygen atoms;
A is a linear or branched alkyl radical comprising from 1 to 8 carbon atoms;
B is a linear or branched alkyl radical, optionally comprising an aromatic ring, comprising from 1. to 9 carbon atoms;
Cₓ is a linear or branched monovalent alkyl radical, in which x indicates the number of carbon atoms, and:
if p is equal to 1, x is from 9 to 25 (9≤x≤25);
if p is equal to 2, x is from 9 to 15 (9≤x≤15),
the ratio i between the number of hydrophobic radicals and the number of glutamic or aspartic units being between 0≤i≤0.5;
when several hydrophobic radicals are borne by a co-polyamino acid, then they are identical or different,
the degree of polymerization DP in glutamic or aspartic units is from 5 to 250;
the free acid functions being in the form of a salt of an alkaline cation chosen from the group consisting of Na⁺ and K⁺.

2. The composition according to claim 1, wherein said hydrophobic radicals are chosen from the hydrophobic radicals according to formula 1 in which p=1, represented by the following formula V:
*(GpR)ᵣ(GpA)ₐGpC Formula V
GpR, GpA, GpC, r and a have the definitions given above.

3. The composition according to claim 1, wherein said hydrophobic radicals are chosen from the hydrophobic radicals according to formula 1 in which a=1 and p=2, represented by the following formula VI:
*(GpR)ᵣGpA (GpC)₂ Formula VI
in which
GpR, GpA, GpC, r and a have the definitions given above.

4. The composition according to claim 1, wherein the co-polyamino acid bearing carboxylate charges and hydrophobic radicals is chosen from the co-polyamino acids of the following formula VII: 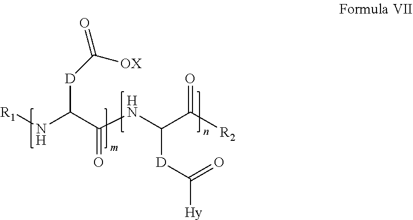
in which,
D represents, independently, either a —CH₂— group (aspartic unit) or a —CH₂-CH₂— group (glutamic unit),
Hy is a hydrophobic radical chosen from the hydrophobic radicals according to formula I or V,
R₁ is a hydrophobic radical chosen from the hydrophobic radicals according to formula I or V′, or a radical chosen from the group consisting of H, a C2 to C10 linear acyl group, a C3 to C10 branched acyl group, benzyl, a terminal “amino acid” unit and a pyrogluamate,
R₂ is a hydrophobic radical chosen from the hydrophobic radicals according to formula I or V in which r=1 and GpR is a radical according to formula II, an —NR′R″ radical, R′ and R″ which are identical or different being chosen from the group consisting of H, the C2 to C10 linear or branched or cyclic alkyls, benzyl, and said alkyl R′ arid R″ may forth together one or more saturated, unsaturated and/or aromatic carbon rings and/or may comprise heteroatoms chosen from the group consisting of O, N and S;
X represents a cationic entity chosen from the group comprising the alkaline cations;
n+m represents the degree of polymerization DP of the co-polyamino acid, that is to say the average number of monomer units per co-polyamino acid chain, and 5≤n+m≤250.

5. The composition according to claim 4, wherein co-polyamino acid bearing carboxylate charges and hydrophobic charges is chosen from the co-polyamino acids according to formula VII, in which R₁═R₁ and R₂═R′₂, of the following formula VIIa: 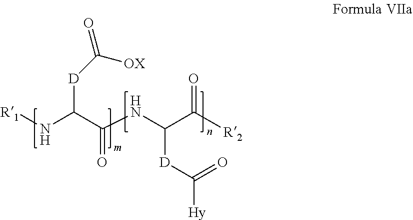
in which
m, n, X, D and Hy have the definitions given above,
R′₁ is a radical chosen from the group consisting of H, a C2 to C10 linear acyl group, a C3 to C10 branched acyl group, benzyl, a terminal “amino acid”' unit and a pyroglutamate,
R′₂ is a —NR′R″ radical, R′ and R″ which are identical or different being chosen from the group consisting of H, the C2 to C 10 linear or branched or cyclic alkyls, benzyl, and said alkyl R′ and R″ may form together one or more saturated, unsaturated and/or aromatic carbon rings and/or may comprise heteroatoms chosen from the group consisting of O, N and S.

6. The composition according to claim 4, wherein the co-polyamino acid bearing carboxylate charges and hydrophobic radicals is chosen from the co-polyamino acids according to formula VII, in which n=0, of the following formula VIIb: 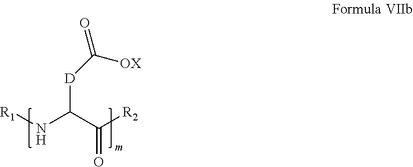
in which m, X, D, R₁ and R₂ have the definitions given above, and at least one R₁ or R₂ is a hydrophobic radical according to formula I or V.

7. The composition according to claim 6, wherein the co-polyamino acid bearing carboxylate charges and hydrophobic radicals is chosen from the co-polyamino acids according to formula VIIb, in which R₇ is a hydrophobic radical according to formula I or V, in which r=1 and GpR is according to formula II.

8. The composition according to claim 4, wherein R₁ is a radical chosen from the group consisting of a C2 to C10 linear acyl group, a C3 to C10 branched acyl group, benzyl, a terminal “amino acid” unit and a pyroglutamate.

9. The composition according to claim 4, wherein R₁ is a radical chosen from the group consisting of a C2 to C10 linear acyl group or a C3 to C10 branched acyl group.

10. The composition according to claim 1, wherein the co-polyamino acid bearing carboxylate charges and hydrophobic radicals is chosen from the co-polyamino acids according to formula VII, VIIa or VIIb: 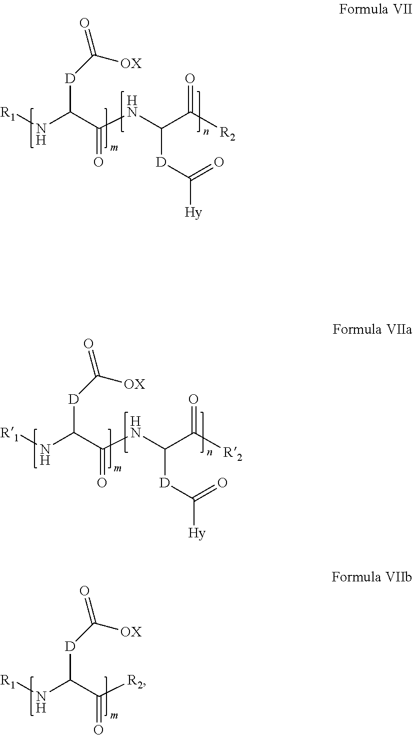
and wherein the group D is a —CH₂— group (aspartic unit).

11. The composition according to claim 1, wherein the co-polyamino acid bearing carboxylate charges and hydrophobic radicals is chosen from the co-polyamino acids according to formula VII, VIIa or VIIb: 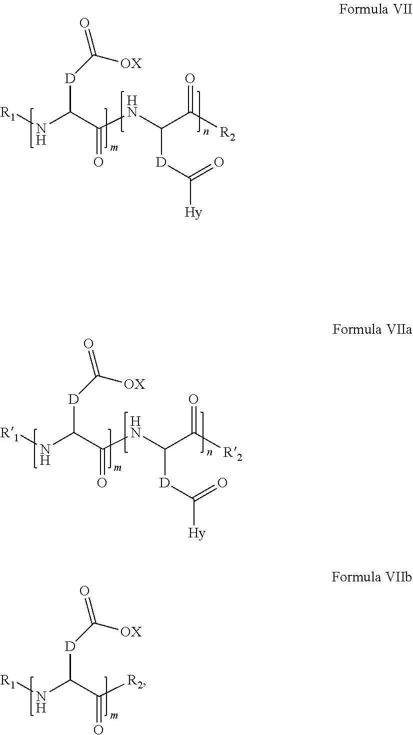
and wherein the group D is a —CH₂-C₂— group (glutamic unit).

12. The composition according to claim 1, wherein the basal insulin of which the isoelectric point is from 5.8 to 8,5 is insulin glargine.

13. The composition according to claim 1, wherein the composition comprises from 40 to 500 U/mL of basal insulin of which the isoelectric point is from 5.8 to 8.5.

14. The composition according to claim 1, wherein the concentration of co-polyamino acid bearing carboxylate charges and hydrophobic radicals is at most 60 mg/mL.

15. The composition according to claim 1, wherein the concentration of co-polyamino acid bearing carboxylate charges and hydrophobic radicals is at most 40 mg/mL.

16. The composition according to claim 1, wherein the concentration of co-polyamino acid bearing carboxylate charges and hydrophobic radicals is at most 20 mg/mL.

17. The composition according to claim 1, wherein the concentration of co-polyamino acid bearing carboxylate charges and hydrophobic radicals is at most 10 mg/mL.

18. The composition according to claim 1, wherein Hy comprises between 20 and 30 carbon atoms.

19. The composition according to claim 1, wherein A is a linear or branched alkyl radical comprising from 1 to 6 carbon atoms.

20. The composition according to claim 1, wherein if p is equal to 1, x is from 11 to 25(11≤x≤25).

21. The composition according to claim 4, wherein Hy is a hydrophobic radical chosen from the hydrophobic radicals according to formula I or V, and in which r=1 and GpR is a radical according to formula II.

22. The composition according to claim 4, wherein R₁ is a hydrophobic radical chosen from the hydrophobic radicals according to formula I or V in which r=0 or r=1 and GpR is a radical according to formula II′, or a radical chosen from the group consisting of H, a C2 to C10 linear acyl group, a C4 to C10 branched acyl group, benzyl, a terminal “amino acid” unit and a pyroglutatnate.

23. The composition according to claim 5, wherein in the co-polyamino acids according to formula VII, R′₁ is a radical chosen from the group consisting of H, a C2 to C10 linear acyl group, a C4 to C10 branched acyl group, benzyl, a terminal “amino acid” unit and a pyroglutamate.

24. The composition according to claim 4, wherein R is a radical chosen from the group consisting of a C2 to C10 linear acyl group, a C4 to C10 branched acyl group, benzyl, a terminal “amino acid” unit and a pyroglutamate.

25. The composition according to claim 4, wherein R₁ is a radical chosen from the group consisting of a C2 to C10 linear acyl group or a C4 to C10 branched acyl group.
